(19) United States
(12) Patent Application Publication
(10) Pub. No.: US 2024/0374819 A1
(43) Pub. Date: Nov. 14, 2024

(54) MEDICAMENT DELIVERY AND SENSING SYSTEM

(71) Applicant: INSULET CORPORATION, Acton, MA (US)

(72) Inventor: Dustin TORGERSEN, Cambridge, MA (US)

(21) Appl. No.: 18/659,431

(22) Filed: May 9, 2024

Related U.S. Application Data
(60) Provisional application No. 63/501,908, filed on May 12, 2023.

(51) Int. Cl. A61M 5/158; A61M 5/172

(52) U.S. Cl. CPC A61M 5/158; A61M 5/1723; A61M 2005/1583; A61M 2005/1726; A61M 2230/201

(57) ABSTRACT

In an aspect, a medicament delivery system is presented. The medicament delivery system includes a housing. The housing includes a medicament delivery device and an injection system. The injection system includes a set of rails. The medicament delivery system includes a first sliding member operable to move on the set of rails and a second sliding member operable to move on the set of rails. The medicament delivery system includes a position element configured to move the first sliding member in a first direction guided by the set of rails and move the second sliding member in a second direction guided by the set of rails.

### CROSS-REFERENCE TO RELATED APPLICATIONS

This application claims priority to and the benefit of U.S. Provisional Application No. 63/501,908, filed May 12, 2023, the entirety of which is incorporated herein by reference.

### TECHNICAL FIELD

The presently disclosed subject matter generally relates to medicament delivery devices. In particular, the presently disclosed subject matter relates to medicament delivery and analyte sensing systems and insertion mechanisms therefor.

### BACKGROUND

Modern drug delivery devices typically have a reservoir that contains a liquid drug, a pump mechanism and an insertion mechanism that introduces a needle or cannula into the subcutaneous region of a user's skin so as to deliver the liquid drug to the user. The user may also use a separate sensing device, such as a glucose sensor, which detects different user physiological attributes by measuring, via a sensing element, changes in the user's blood chemistry via the subcutaneous region of the user's skin. The drug delivery device and the sensing devices are located apart due to the potential for interference in both the delivery of the liquid drug as well as problems with the sensing element caused by puncture wounds to the user's skin from the delivery element and also interference by the liquid drug on the sensing element.

### SUMMARY OF THE DISCLOSURE

In an aspect, a medicament delivery system is presented. The medicament delivery system includes a housing. The housing includes a medicament delivery device and an injection system. The injection system includes a set of rails. The medicament delivery system includes a first sliding member operable to move on the set of rails and a second sliding member operable to move on the set of rails. The medicament delivery system includes a position element configured to move the first sliding member in a first direction guided by the set of rails and move the second sliding member in a second direction guided by the set of rails.

In another aspect, a medicament delivery apparatus is presented. The medicament delivery apparatus includes a medicament delivery device and a set of rails. A gap is between individual rails of the set of rails. The medicament delivery apparatus includes an injection device positioned adjacent to the gap of the set of rails and positioned to mirror the sensing element. The medicament delivery apparatus includes a biasing mechanism coupled to a first sliding member. The first sliding member is operable to move a cannula along the set of rails in a first direction and a sensing element along the set of rails in a second direction, opposite the first direction. The medicament delivery apparatus includes an insertion funnel positioned opposite the set of rails, wherein the insertion funnel includes an insertion funnel entrance configured to allow a passage of the sensing element into the insertion funnel.

In another aspect, a medicament delivery system is presented. The medicament delivery system includes a housing. The housing includes a medicament delivery device. The medicament delivery device includes a pump. The medicament delivery device includes an injection system connected to the pump. The injection system includes a first set of rails and a second set of rails. The medicament delivery system includes a first sliding member operable to move on the first set of rails, the first sliding member including a sensor. The medicament delivery system includes a second sliding member operable to move on the second set of rails, the second sliding member including a cannula connected to the pump. The medicament delivery device includes a positioning element configured to move the first sliding member in a first direction guided by the first set of rails and move the second sliding member in a second direction guided by the second set of rails.

These and other aspects and features of non-limiting embodiments of the presently disclosed and claimed subject matter will become apparent to those skilled in the art upon review of the following description of specific non-limiting embodiments of the disclosed subject matter in conjunction with the accompanying drawings.

### BRIEF DESCRIPTION OF THE DRAWINGS

FIG. 1A illustrates a block diagram of an example of a wearable medicament delivery and sensing system.

FIGS. 1B-1D illustrates an exemplary embodiment of an insertion system;

FIGS. 2A-2F illustrates another exemplary embodiment of an insertion system;

FIGS. 2G-21 illustrate yet another exemplary embodiment of an insertion system;

FIG. 3A-3C illustrates yet another exemplary embodiment of an insertion system;

FIGS. 4A-4I illustrates a further exemplary embodiment of an insertion system; and

FIG. 5 illustrates an exemplary embodiment of a wearable medicament delivery and sensing system.

### DETAILED DESCRIPTION

In the following description, for the purposes of explanation, numerous specific details are set forth in order to provide a thorough understanding of the presently disclosed subject matter. It will be apparent, however, that the presently disclosed subject matter may be practiced without these specific details. As used herein, the word “exemplary” or “illustrative” means “serving as an example, instance, or illustration.” Any implementation described herein as “exemplary” or “illustrative” is not necessarily to be construed as preferred or advantageous over other implementations. All of the implementations described below are exemplary implementations provided to enable persons skilled in the art to make or use the embodiments of the disclosure and are not intended to limit the scope of the disclosure, which is defined by the claims.

At a high level, aspects of the present disclosure are related to medicament delivery and sensing devices and systems of medicament delivery and sensing devices. A medicament delivery and sensing system may include a mechanism that enables the insertion of a sensing element of a sensor into a first location in the epidermis of a user and the insertion of a needle and/or cannula for liquid medicament delivery in a second location in the user's epidermis, both of which may be automated or manually triggered. There may be some risk in locating a cannula too close in proximity (e.g., <1 inch or less than 25.4 millimeters (mm)) to a sensor (e.g., interference with the sensor by delivery of the liquid, additional fluids generated by the body to repair the wound caused by puncturing the epidermis, and the like). Additionally, damage to the epidermis due to puncture wounds from an introducer (needle/trocar) may lead to increased sensor warmup times. Some of the examples described herein factor in a distance metric and provide both introducer and introducer-less sensor insertion options. The examples of the present disclosure can be used to provide a sensing device co-located with a medicament delivery device in a wearable medicament delivery and sensing system.

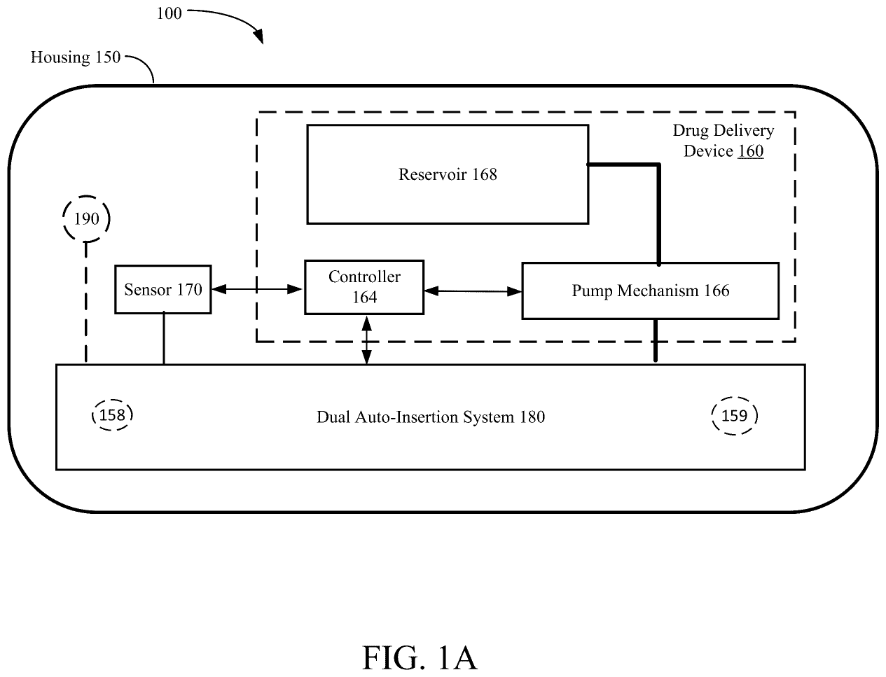

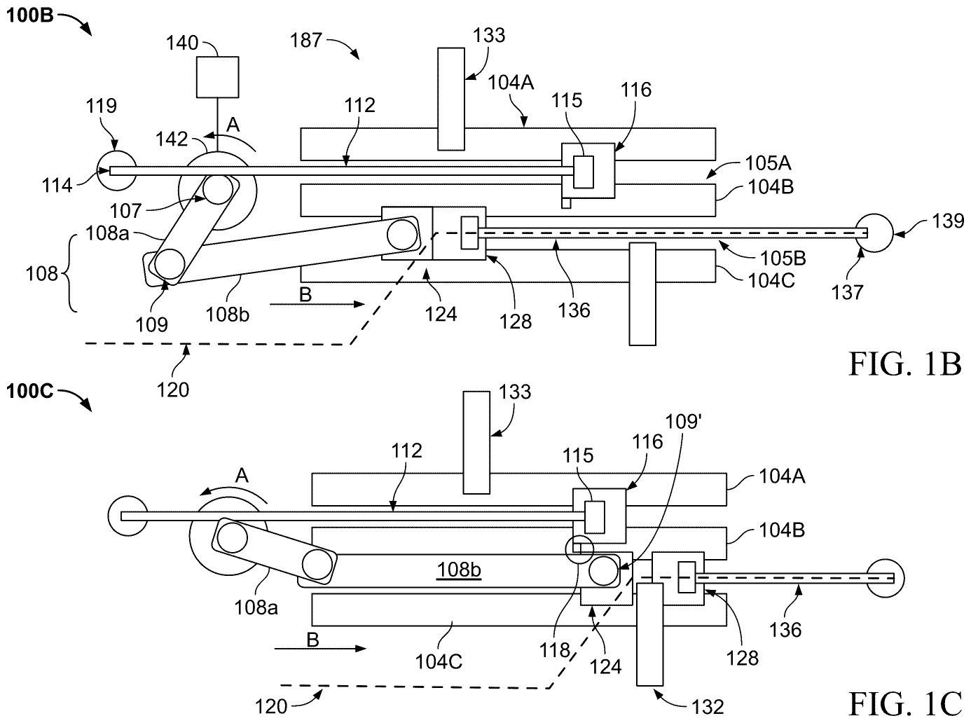

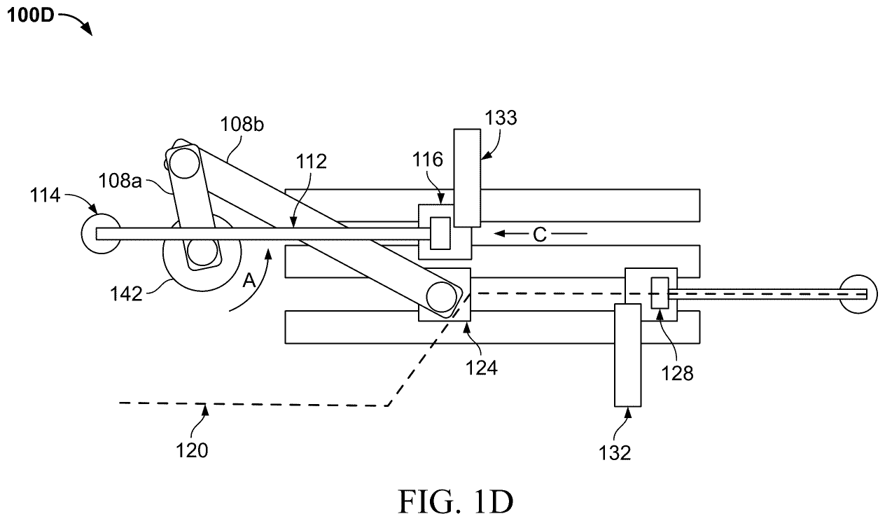

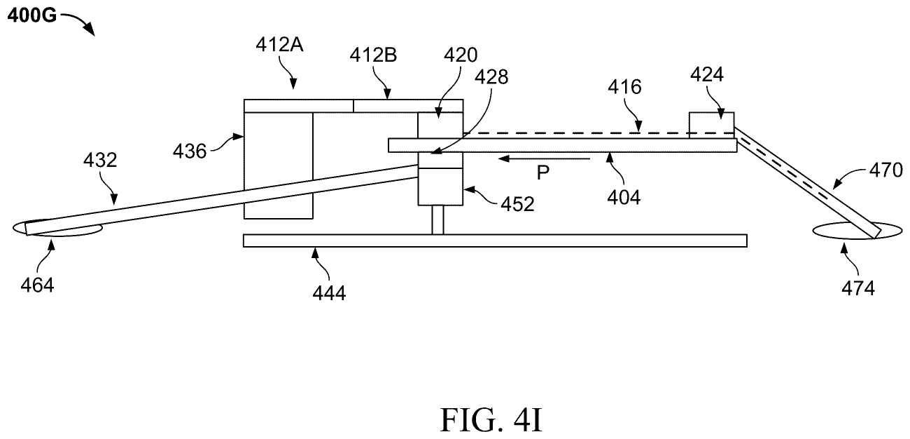

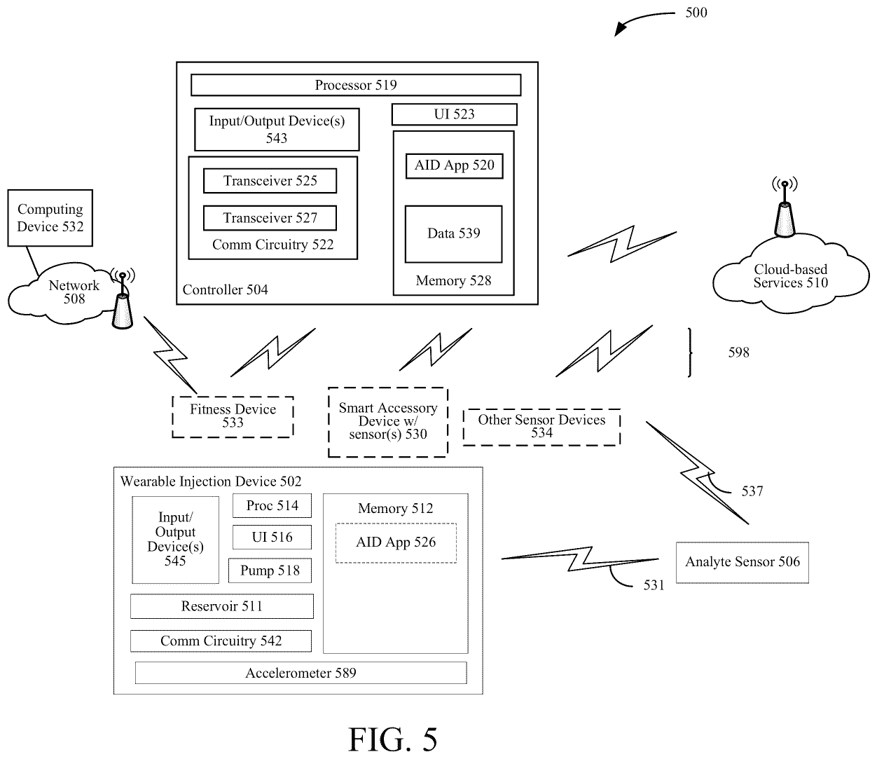

FIG. 1A illustrates a block diagram of an example of a wearable medicament delivery and sensing system. The wearable medicament delivery and sensing system 100 may include a housing 150, which contains a medicament delivery device 160, a sensor 170, and an insertion system 180. The housing 150 may have multiple parts and/or housings that fit together to provide a single device in appearance that is attachable to the skin of a user. For example, the housing 150 may have a first housing portion that contains one or more of the components described herein and is attachable to the skin of a user with adhesive and a second housing portion that contains one or more of the components described herein, and which may also be attachable to the skin of a user with an adhesive. Additionally, or alternatively, the first and second housing portions may fit together in a tray or cradle, which is adhered to the user's skin with an adhesive. The housing 150 may have housing exits, such as 158 and 159, that are configured and sized to enable a sensing device or element (described later) of sensor 170 and a needle/cannula (described later) to exit the housing 150 and penetrate into the skin of a user. The medicament delivery device 160 may include a controller 164, a pump mechanism 166, and a reservoir 168. The controller 164 may be operable to communicate or provide/receive signals to/from the sensor 170, the insertion system 180, and/or the pump mechanism 166, or other elements as explained with reference to FIG. 5. For example, the controller 164 may be operable to cause delivery of a liquid medicament from the reservoir 168 via the pump mechanism 166 and a fluid pathway created by the insertion system 180 based on measurements provided by the sensor 170. In addition, the controller 164 may be operable to activate or actuate the insertion system 180 as described with reference to the following examples. The reservoir 168 may be operable to contain a liquid drug or medicament that may be or include any medicament in liquid form capable of being administered by the medicament delivery device via a subcutaneous cannula, including, for example, insulin, glucagon-like peptide-1 (GLP-1), glucose-dependent insulinotropic polypeptide (GIP), pramlintide, glucagon, co-formulations of two or more of GLP-1, GIP, pramlintide, and insulin; as well as pain relief medicaments, such as opioids or narcotics (e.g., morphine, or the like), methadone, blood pressure medicines, chemotherapy medicaments, fertility medicaments, or the like.

The housing 150 may also include an opening for a mechanical actuator 190 (e.g., a button, a sliding mechanism, a dial, or the like) that is coupled to the insertion system 180. The mechanical actuator 190 may be configured to receive an input from outside the housing 150 (e.g., from a user's finger or hand) and, in response to the input, the insertion system 180 may actuate as described with reference to the following examples. Alternatively, actuation of the insertion system 180 may be automated or triggered by a wireless communication from a separate controller, as explained elsewhere herein.

Examples of medicament delivery devices are described, for example, in U.S. Pat. Nos. 7,128,727; 7,018,360; 7,144,384; and 10,420,883 and U.S. Patent Application Publication Nos. 2007/0118405, 2006/0282290, 2005/0238507, and 2004/0010207, which are incorporated herein by reference in their entirety.

In some embodiments, aspects of the present disclosure can be applied to existing medicament delivery device mechanisms to reduce cost of manufacturing or the number of elements or mechanisms required for insertion of a medicament delivery element and a sensing element. An insulin pump co-located with a glucose sensing device has long been a goal of the diabetes industry. Packaging these two pieces of technology together, coupled via an insertion system, would be beneficial and advantageous from a user-experience perspective as well as enabling the integration of a sensor and a medicament delivery device within a single device or at a single location.

FIG. 1B illustrates an exemplary embodiment of an insertion system usable in the exemplary medicament delivery and sensing system 100 shown in FIG. 1A. The insertion system 187 of FIG. 1B is shown in an initial or pre-deployment position. The insertion system 187 of FIG. 1B may include a number of rails, such as rails 104A, 104B and 104C, in this example. A “rail” as used in this disclosure is one or more structural elements that guide one or more objects in a direction. Rails 104A, 104B and 104C may include one or more materials, such as, but not limited to, plastic, metal, and the like. Rails 104A-C may be oriented in a horizontal direction, vertical direction, and/or combination thereof, without limitation. There may be gaps between the rails 104A, 104B and 104C that allow passage of one or more elements between and/or along the rails 104A-C. A first rail 104A may be adjacent to the first horizontal gap 105A on a first side of the first horizontal gap 105A, and a second rail 104B may be adjacent to the first horizontal gap 105A on a second side of the first horizontal gap 105A. The first rail 104A and the second rail 104B may form a first pair of rails. The second rail 104B may be adjacent to a second horizontal gap 105B that is between the second rail 104B and a third rail 104C. The second rail 104B and 104C may form a second pair of rails.

The insertion system 187 may include a sensing device or element 112. A “sensing device” or “sensing element” as used in this disclosure is a device capable of detecting analytes in the blood or liquids in the interstitial tissue of the user. Sensing element 112 may include a blood glucose sensor, such as a continuous glucose monitor (CGM), a ketone sensor, a blood oxygen sensor, or the like. In an example, the sensing element 112 may be configured to detect one or more blood glucose values of a user and communicate the one or more blood glucose values, or data indicative of glucose values, to a computing device, such as a controller 164 of FIG. 1A.

The sensing element 112 may be guided by rails 104 with an insertion end 114 of the sensing element 112 extending beyond a biasing mechanism 142 on one side and a driven end 115 extending beyond the rails 104B and 104C at an opposite side. Of course, the orientation may be reversed. An “insertion end” as used in this disclosure is a part of an object that is inserted into a user. An insertion end 114 of sensing element 112 may include a needle, cannula, trocar, and/or other piercing element. In some embodiments, the needle, cannula, or trocar of the sensing element 112 may be hollow and the sensing element 112 may be within the needle, cannula, or trocar. For instance, and without limitation, sensing element 112 may include an insertion end including a piercing element on an end of a hollow tube and a head end including a sensor or other device connected to the hollow tube opposite the piercing element. The insertion end 114 may exit the housing 150 at housing exit 119.

The insertion system 180 may include needle/cannula 136. Needle/cannula 136 may be configured to provide a fluid pathway from the reservoir to a user for delivery of the liquid medicament from the reservoir of the medicament delivery device. Needle/cannula 136 may include an insertion end 137 on a side (e.g., the right side in FIG. 1B) of rails 104B and 104C. In some embodiments, needle/cannula 136 may be configured to move in a linear or curvilinear direction with respect to rails 104, such as a forward direction represented by direction arrow B as well as a backward or retraction motion shown in a later example. In some embodiments, needle/cannula 136 may include a hollow tube portion connected to a fluid reservoir. The needle/cannula 136 may be connected to fluid pathway 120, which is coupled to the reservoir 168. The fluid pathway 120 may include, without limitation, tubing formed from one or more of steel, plastic, polyvinyl chloride (PVC), or the like. In some embodiments, the fluid pathway 120 fluidly couples with the needle/cannula 136 at the sliding member 128. In an exemplary embodiment, fluid pathway 120 comprises a needle; and a cannula comprises member 136. The fluid coupling between the fluid pathway 120 and the cannula 136 is leakproof. In an exemplary embodiment, fluid pathway 120 (e.g., a needle) may be located inside cannula 136 and may slide within cannula 136. The housing exit 139 may be configured to guide the fluid pathway 120 and cannula 136 into the skin at an appropriate angle and depth to ensure successful delivery of the liquid medicament to the user. Similarly, the housing exit 137 may be configured to guide the sensor device 112 into the skin at an appropriate angle and depth to ensure sensing of the user's analytes.

Still referring to FIG. 1B, the insertion system 180 may include linkage mechanism 108. A “linkage mechanism” as used in this disclosure is an object configured to push and/or pull, directly or indirectly, one or more other objects. Linkage mechanism 108 may include, without limitation, a spring, a lever(s), arm(s), and/or other mechanism. In some embodiments, linkage mechanism 108 includes a biasing mechanism 142, such as a torsion spring. The biasing mechanism 142 may be configured to rotate in a clockwise or counter-clockwise direction. FIG. 1B shows the biasing mechanism 142 in a pre-deployment position, or an initial position. The biasing mechanism 142 may be actuated by trigger 140. Trigger 140 may include, but is not limited to, one or more wires, piezoelectric elements, and/or shape-memory alloys as well as a number of mechanical features, such as latches, rods, detents, gears, or the like that may be configured to hold or release the biasing mechanism 142 in or from a “loaded” position. A loaded position may be the position in which the biasing mechanism 142 has sufficient potential energy to drive linkage mechanism 108 causing the insertion system to deploy. For instance, and without limitation, trigger 140 may include a shape memory alloy (SMA) wire that may activate or otherwise enable release of the biasing mechanism 142. An SMA wire may be attached to an actuating element. Alternating actuations or pulses of the SMA wire may drive the actuating element to pivot back and forth. The actuating element may turn a ratchet gear that may, in turn, drive a leadscrew or a reciprocating element that causes liquid medicament to flow out of a reservoir of the medicament delivery and sensing system 100. In some embodiments, a release bar may be coupled to or in contact with a portion of the ratchet gear. The release bar may be moved out of an original or loaded position after one or a few actuations of the actuating element, in some embodiments. The release bar may release potential energy and turn out of a loaded position which may allow a spring or a biasing mechanism of the insertion system to fire. In some embodiments, an SMA wire or other actuating element may be configured to trigger a release mechanism that may allow a spring to fire. A spring may be connected to linkage mechanism 108 and/or biasing mechanism 142. In some embodiments, an SMA wire may trigger biasing mechanism 142 directly. Linkage mechanism 108 may include one or more arms. For instance, and without limitation, linkage mechanism 108 may include a first arm 108a and a second arm 108b. The first arm 108a of linkage mechanism 108 may be coupled at one end to the biasing element 142 via coupling point 107. The first arm 108a and the second arm 108b are movably connected at rotatable joint 109. The coupling of the first arm 108a and the second arm 108b of linkage mechanism 108 at joint 109 may be by, but not limited to, one or more rivets, screws, bolts, clips, and the like. The second arm 108b of the linkage mechanism 108 may be configured to move in a linear or curvilinear movement forwards (as shown by direction arrow B) and/or backwards (as described later with reference to FIG. 1C) along the gap 105B.

In some embodiments, linkage mechanism 108 may be coupled to a sliding member 124. Sliding member 124 may be positioned at an end of the second arm 108b of linkage mechanism 108. The sliding member 124 may be coupled to the second arm 108b via a rotatable joint 109′.

The sensing element 112 may be coupled to a sliding member 116 that may be coupled to a sensing element 112 and may be positioned opposite the insertion end 114 of 112. The sliding member 116 may be operable to slide upon rails 104A and 104B and within the gap 105A, which allows the sliding member 116 to be securely guided along the rails 104A and 104B. The sensing element 112 may be positioned adjacent to the gap 105A.

Operationally, FIG. 1B illustrates an initial or pre-deployment position of the insertion system 180.

Referring now to FIG. 1C, a deployment operation of the insertion system 180 is shown. The trigger 140 may be coupled to a controller, such as 164, to a mechanical actuator, such as 190, or both. To cause the insertion system 180 to deploy, the trigger 140 may receive a release signal or an input causing the trigger to release the potential energy of the biasing mechanism 142. In response to the release signal or input, the biasing mechanism 142 may begin to rotate in direction A. In response to a rotational force from biasing mechanism 142, for example, in the direction shown by rotation arrow A, the first arm 108a of linkage mechanism 108 (and joint 109) may be configured to rotate in the direction of arrow B, pushing the second arm 108b (and joint 109′) also in direction of arrow B. The second arm 108 may push both sliding member 124 and sliding member 128, a part of which is positioned within gap 105B, along rails 104B and 104C toward the housing exit 139. The cannula 136 may be positioned adjacent to the gap 105B.

In more detail, the rotation of the biasing mechanism 142 causes the linkage mechanism 108 fixed at joint 107 to move in the direction shown by direction arrow B. The rotatable joint 109 that couples the first arm 108a to the second arm 108b advances, enabling the first arm 108a to move the second arm 108b along gap 105B. The moving of the second arm 108b along the gap 105B also pushes the sliding member 124 and sliding member 128 along the gap 105B.

The sliding member 128 is coupled to the fluid pathway 120 and the cannula 136. The fluid pathway 120 has sufficient elasticity, flexibility, slack, or a combination thereof to follow the cannula sliding member 128 as the second arm 108b causes the cannula sliding member 128 to traverse along rails 104B and 104C. As the sliding member 128 is pushing by sliding member 124 and the second arm 108b, the cannula 136 and the fluid pathway 120 are directed toward the housing exit 139 and the insertion end 137 exits the housing exit 159 with sufficient force into puncture the skin of the user and travel an appropriate distance or depth within the skin for subcutaneous delivery of the liquid medicament. An appropriate distance may include a range between, but not limited to, about 1 mm to about 8 mm, and may preferably be about 5 mm. In other embodiments, an appropriate distance may be greater than 5 mm or less than 5 mm, without limitation.

To ensure the cannula 136 is held at the appropriate depth in the skin, sliding member 128 may be held in place and prevented from retracting by catch block 132. The catch block 132 may be configured to resist the movement of sliding member 128 in the direction opposite to the direction indicated by direction arrow B, and may also be configured to slow movement of sliding member 128 in the direction indicated by arrow B when inserting cannula 136. The catch block 132 may be made from a material such as, but not limited to, plastic, rubber, a damping material, and the like. Catch block 132 may slow down motion of the cannula 136 and fluid pathway (e.g., needle) 120 during insertion, such that the insertion speed through the skin of the user at the end of the insertion stroke may be slower than the insertion speed through the skin at the beginning of the insertion stroke. Catch block 132 may cause this slowing down to occur by frictionally engaging or dampening motion of sliding member 128 as it passes by catch block 132, thereby slowing down but not stopping the insertion process of cannula 136 in the forward direction (e.g., in the direction of arrow “B”). Accordingly, catch block 132 may be referred to as “slowing member” 132 in some exemplary embodiments or in a portion of its operation, in that it slows down motion of sliding member 128, but at the same time can block sliding member 128 from retracting backward (e.g., in the direction opposite arrow “B”). In this sense, catch block 132 may serve a dual purpose of slowing down motion of sliding member 128 in a first direction, and stopping motion of sliding member 128 in another direction opposite to the first direction. Catch block 132, when serving as a slowing or dampening member, may slow insertion of cannula 136 at the end of its stroke (e.g., at the last ½, ¼, or ⅛ of its insertion stroke), and may slow the insertion process of cannula 136 down by, for example, 2 ms, 5 ms, 10 ms, 20 ms, 50 ms, or 100 ms. In an example, catch block 132 may be affixed to one or more rails of rails 104 by adhesive, welding, screws, or the like. Catch block 132 may secure sliding member 128 at a predetermined position along rails 104B and 104C and cannula 136 at a predetermined insertion distance (e.g., beyond a bottom surface of housing 150). For instance, and without limitation, catch block 132 may secure cannula 136 such that its distal end extends approximately 3-10 millimeters (mm) from a bottom surface of housing 150, or in some embodiments, about mm to about 30 mm from a bottom surface of housing 150.

During the deployment of linkage mechanism 108 and the sliding of sliding member 124 and sliding member 128, the sliding member 124 may connect to the sliding member 116, such as through one or more arms, protrusions (shown generally as 118), and the like of sliding member 116.

For example, the sliding member 116 may include one or more protrusions, such as 118, that may interact with one or more other elements, such as a notch or detent formed in or on sliding member 124, or an end of sliding member 124, and or damping block 133. For instance, and without limitation, sliding member 116 may include a protrusion extending towards gap 105B and rail 104C that causes sliding member 116 to engage sliding member 124. During deployment of cannula 136 as shown in the example of FIG. 1C, sliding member 124 and sliding member 128 may be configured to slide past the protrusion 118 of sliding member 116 as the cannula 136 is being deployed or inserted. The protrusion 118 of sliding member 116 may be configured to engage sliding member 124, that is coupled to the second arm 108, upon retraction of sliding member 124 and fluid pathway (e.g., needle) 120 in the direction opposite arrow “B.” This may be considered the end phase of deployment of cannula 136.

After the end phase of cannula deployment is completed, the biasing mechanism 142 continues to rotate in the direction A, which may be considered the beginning of a retraction operation of second arm 108b and sliding member 124, and deployment of sensing element 112.

FIG. 1D illustrates the retraction operation of the insertion system 180. Linkage mechanism 108, in response to the rotational force provided by biasing mechanism 142, may be configured to retract the first arm 108a and the second arm 108b from the extended position. The catch block 132 is configured to secure or catch sliding member 128 such that cannula 136 remains in a deployed position. As shown in FIG. 1D with reference to FIG. 1C, the first arm 108a continues rotating in direction “A” around the joint 107 that couples the first arm 108a to biasing mechanism 142. The first arm 108a and the second arm 108b continue to rotate around the joint 109 that couples the first arm 108a to the second arm 108b, which enables the first arm 108a to pull the second arm 108b in direction C, opposite the direction arrow B. Direction C may be referred to as the retraction direction or the sensor deployment direction. As the first arm 108a and second arm 108b retract from the extended position, sliding member 124 may be configured to pull sliding member 116, which may be coupled to a sensing element 112, towards housing exit 114 for sensing element 112. Sliding member 116 may be guided toward housing exit 114 by rails 104A and 104B and gap 105A. The biasing mechanism 142 applies sufficient force in direction C to the sensing element 112 to drive the sensing element 112 into the subcutaneous region of the user's skin to a depth sufficient to enable the sensing element 112 to obtain accurate measurements of the analytes that the sensing element 112 is configured to measure. Through retraction of the linkage mechanism 108, sensing element 112 may be deployed through movement of sliding member 124 connected to sliding member 116. Linkage mechanism 108 may be configured to deploy cannula 136 through an extension movement and deploy sensing element 112 through a retraction movement in one seamless movement.

As explained above with reference to catch block 132, an additional catch or dampening member may be employed when inserting sensing element 112. For example, to ensure sensing element 112 is held at the appropriate depth in the skin, sliding member 116 may be held in place and prevented from retracting by catch block 133. The catch block 133 may be configured to resist the movement of sliding member 116 in the “B” direction, and may also be configured to slow movement of sliding member 116 for at least a portion of its movement in the direction indicated by arrow “C” when inserting sensing element 112. The catch block 133 may be made from a material such as, but not limited to, plastic, rubber, a damping material, and the like. Catch block 133 may slow down motion of the sensing element 112 during insertion, such that the insertion speed through the skin of the user at the end of the insertion stroke may be slower than the insertion speed through the skin at the beginning of the insertion stroke. Catch block 133 may cause this slowing down to occur by frictionally engaging or dampening motion of sliding member 116 as it passes by catch block 133, thereby slowing down but not stopping the insertion process of sensing element 112 in the forward direction (e.g., in the direction of arrow “C”). Accordingly, catch block 133 may be referred to as “slowing member” or “dampening member” 133 in some exemplary embodiments or in a portion of its operation, in that it slows down motion of sliding member 116, but at the same time can block sliding member 116 from retracting backward (e.g., in the direction of arrow “B”). In this sense, catch block 133 may serve a dual purpose of slowing down motion of sliding member 116 in a first direction, and stopping motion of sliding member 116 in another direction opposite to the first direction. Catch block 133, when serving as a slowing or dampening member, may slow insertion of sensing element 112 at the end of its stroke (e.g., at the last ½, ¼, or ⅛ of its insertion stroke), and may slow the insertion process of sensing element 112 down by, for example, 2 ms, 5 ms, 10 ms, 20 ms, 50 ms, or 100 ms. In an example, catch block 133 may be affixed to one or more rails of rails 104 by adhesive, welding, screws, or the like. Catch block 133 may secure sliding member 116 at a predetermined position along rails 104B and 104C and sensing element 112 at a predetermined insertion distance (e.g., beyond a bottom surface of housing 150). For instance, and without limitation, catch block 133 may secure sensing element 112 such that its distal end extends approximately 3-10 millimeters (mm) from a bottom surface of housing 150.

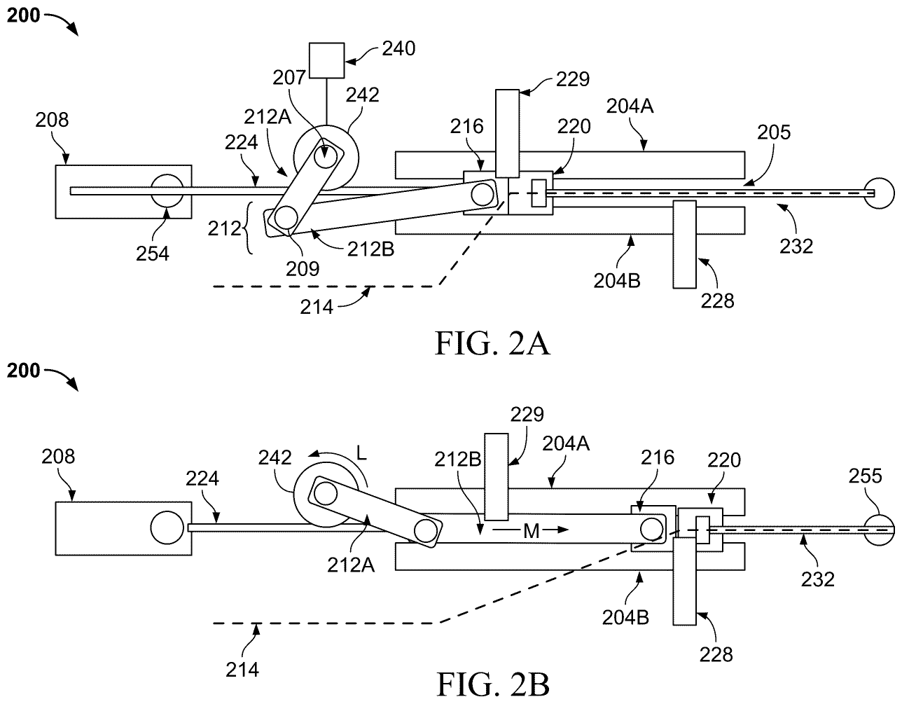

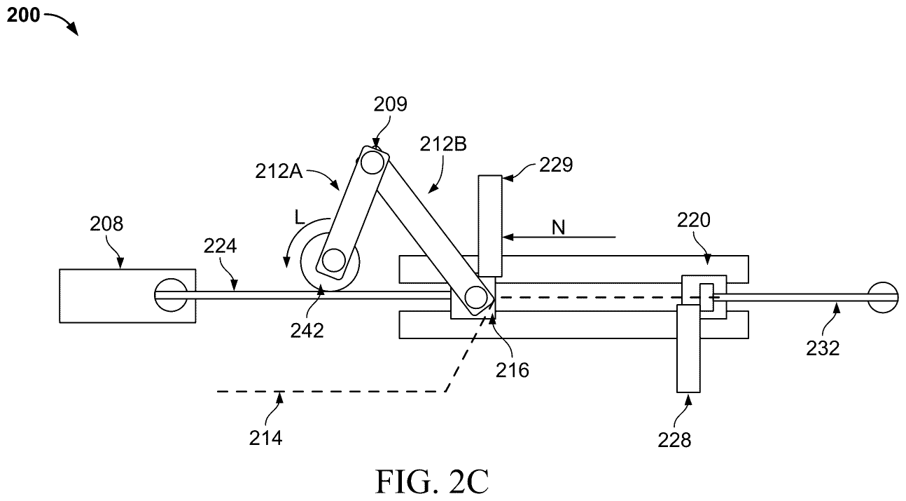

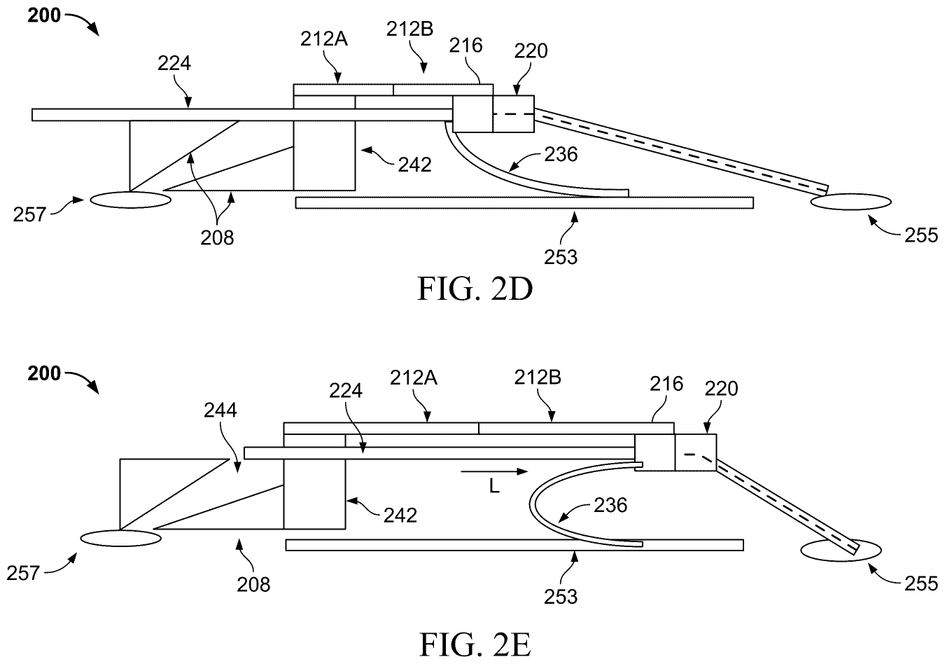

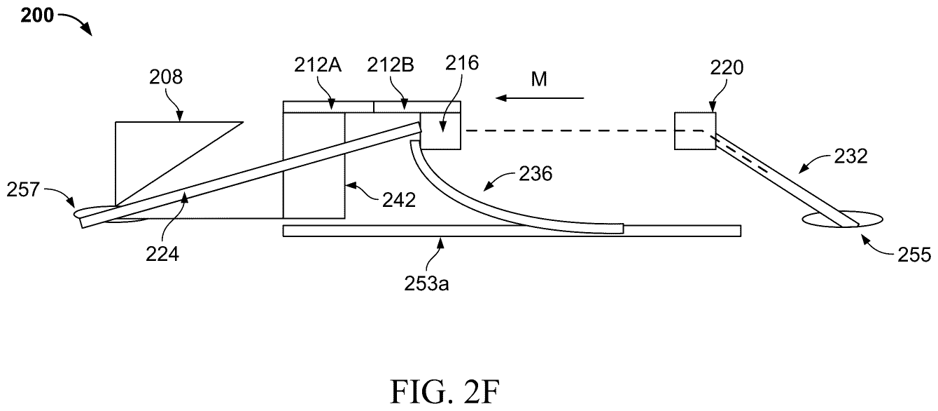

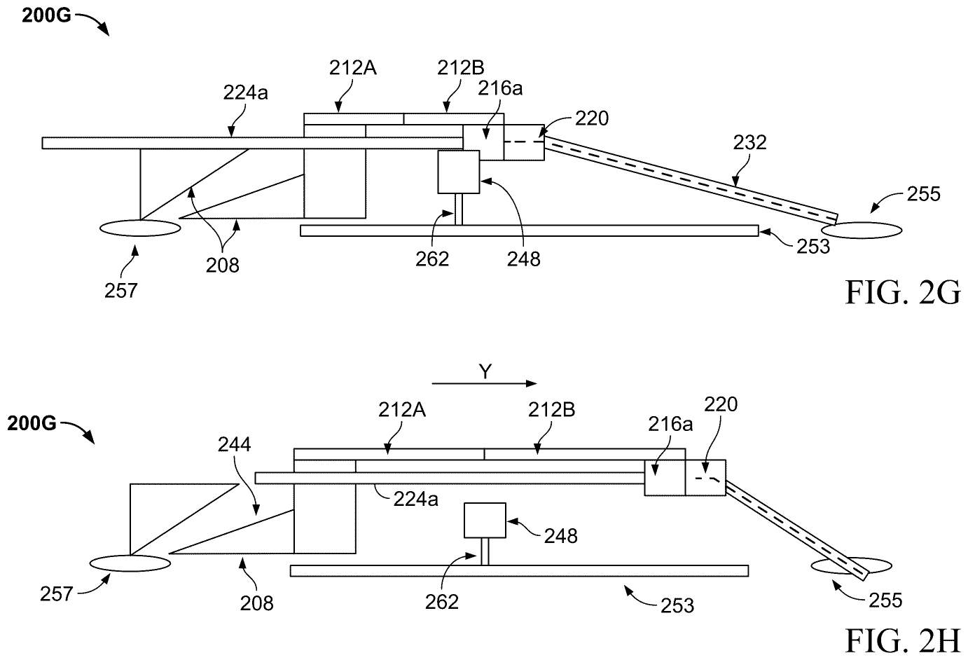

Referring now to FIG. 2A, another embodiment of an insertion system 200 is shown. The insertion system 200 may be usable as the insertion system 180 in the medicament delivery system 100 described above with reference to FIG. 1A. For example, insertion system 200 may include a trigger 240, a catch block 228, catch block 229, a sliding member 220, fluid pathway 214 (which may comprise a needle or other conduit), and/or cannula 232, each of which may be similar in structure and function as corresponding components described above with reference to FIGS. 1B-1D. The insertion system 200 may include a sensing element 224 that may function in a manner similar to the sensing elements described above with reference to FIGS. 1A-1D. In addition, the cannula 232 of insertion system 200 may be coupled to a reservoir (e.g., via fluid pathway 214, which may comprise a needle) configured to contain a liquid medicament.

The insertion system 200 may include rails 204A and 204B. Rails 204A and 104B may be structurally similar to rails 104 as described above with reference to FIGS. 1B-1D, without limitation. In some embodiments, rails 204A and 204B may be coupled together to form a singular rail. Rails 204A and 204B may be separated by a gap 205 that is configured to allow passage for sliding member 216 and the sliding member 220 to move forward and/or backward along rails 204A and 204B. A sensing element 224 may be coupled to sliding member 216 and a cannula 232 may be coupled to the sliding member 220. The coupling between the cannula 232 and the sliding member 220 is operable to enable the cannula to move with the sliding member 220. The fluid pathway 214 is a conduit that couples the reservoir (not shown in this example) to the cannula 232 and may comprise a needle or other cylindrical conduit. The cannula 232 may be fluidly coupled to the fluid pathway 214 via a leak-proof coupling at or about the sliding member 220.

The linkage mechanism 212 may be similar to that of linkage mechanism 108 above. Similar to the arrangement in the example of FIGS. 1B-1D, the linkage mechanism 212 may couple to the biasing element 242 via joint 207. The linkage mechanism 212 may include a first arm 212a and a second arm 212b that are coupled together at joint 209. The linkage mechanism may be coupled to the biasing element 242.

The biasing element 242, which may be a torsion spring or the like, may store potential energy that may be released by trigger 240. Trigger 240 may be configured and function in a manner similar to that of trigger 140 of FIG. 1B.

In a further embodiment, the insertion system 200 may also include insertion funnel 208 that may be formed from a material such as, but not limited to, plastic, metal, and the like. The insertion funnel 208 may include an insertion funnel entrance 254 that may lead to a structure that may guide sensing element 224 toward a housing exit. For instance, and without limitation, the insertion funnel entrance 254 may be an opening with a slanted wall or walls that may direct the sensing element 224 downwards out of a housing exit for insertion into the skin of the user.

FIG. 2A illustrates a top view of another exemplary insertion system. A pre-deployment or initial position of the insertion system 200 is illustrated in FIG. 2A. The biasing mechanism 242 may be configured in the same manner as biasing mechanism 142 as described with reference to the example of FIGS. 1B-1D. The trigger 240 may be operable to release the biasing mechanism 242 in response to receiving an input (in a manner similar to that described above with reference to FIGS. 1B-1D).

Referring now to FIG. 2B, a deployment operation of system 200 is illustrated with reference to a top view of system 200. The biasing mechanism 242 may be activated and may begin to rotate in in a counter-clockwise motion or L direction as shown direction arrow L. The rotation in the L direction may cause the first arm 212a and/or second arm 212b of positioning element 212 to extend. The second arm 212b of the positioning element 212 may be attached to sliding member 216 of sensing element 224. Extension of the second arm 212b may cause sliding member 216 to be pushed against sliding member 220 of injection device 232. As the second arm 212b is extended, sliding member 216 and the sliding member 220 are guided in the direction M (shown by direction arrow M) by gap 205 along the rails 204A and 204B that forces the cannula 232 into the housing exit 255. The fluid pathway 214 has sufficient elasticity, flexibility and/or slack to allow the fluid pathway 214 to move with the sliding member 220 and remain fluidly coupled to the cannula 232. An insertion end (not shown in this example) of the cannula 232 is operable to penetrate the skin of the user to an appropriate depth to permit the delivery of the liquid medicament.

As the second arm 212B reaches its full extension, the catch block 228 may be operable to lock the second sliding block 220 in place which prevents the sliding member 220 from retracting back toward the biasing mechanism 242 and also assists in maintaining the cannula 232 in position within the skin of the user. Catch block 228 may also serve to slow down insertion of cannula 232 during at least a portion of its insertion phase in a manner similar to that described above with reference to FIGS. 1B-1D.

While the second arm 212B has reached its full extension, the biasing member 242 continues rotating to retract sliding member 216 and deploy the sensing element 224.

Also, during the deployment of the cannula 232, the sensing element 224 is pulled over the insertion funnel 208 and past the insertion funnel entrance 254. This prepares the insertion system 200 to deploy the sensing element 224 during a retraction operation of the insertion system 200.

Referring now to FIG. 2C, a retraction operation of insertion system 200 is illustrated. As the biasing member 242 continues to cause the first arm 212a of the positioning element 212 to rotate in a counter-clockwise direction (direction L), the first arm 212a pulls the second arm 212B in direction N, which is the direction opposite to direction M of FIG. 2B. As shown in FIG. 2C, the first arm 212a and the second arm 212b rotate about joint 209 and sliding member 216 that is coupled to the end of the second arm 212b is also pulled in the direction N. As sliding member 216 begins its motion in the direction N, an end of the sensing element 224 may fall into the insertion funnel entrance 254 as a pre-insertion position relative to insertion funnel 208. As the biasing mechanism 242 continues to cause rotation in the L direction, sliding member 216 drives the sensing element 224 into the insertion funnel 208 for exiting a housing exit (not shown in this example) and puncturing the skin of the user.

FIG. 2D illustrates a side view of the exemplary embodiment of system 200 shown in FIG. 2A. FIG. 2D shows a pre-deployment operation of system 200. System 200 may include wire 236. Wire 236 may include, but is not limited to, a copper, silver, or other wire. The wire 236 may be configured to provide an electrical connection between the sensing element 224 and the circuitry 253. The circuitry 253 may include one or more resistors, capacitors, transistors, inductors, and the like. The circuitry 253 may include a printed circuit board (PCB). The wire 236 may be connected to sliding member 216 of sensing element 224. Sensing element 244 may be positioned above the insertion funnel 208 in a pre-deployment stage. The insertion funnel 208 may include a rigid structure that may be composed of plastic, and/or other materials. The insertion funnel 208 may comprise a first wall and a second wall. The first wall of the insertion funnel 208 may be shaped as a right triangle. In some embodiments, the first wall of the insertion funnel 208 may be shaped as a square, rectangle, or other shape. The first wall of the insertion funnel 208 may be positioned approximate the biasing mechanism 242. In some embodiments, a distal end of the sensing element 244 may rest on a top surface of insertion funnel 208. For instance, a left distal end of the sensing element 244 may rest on a left wall of insertion funnel 208. The left wall of the insertion funnel 208 may be positioned approximate the insertion funnel entrance 254. The left wall of the insertion funnel 208 may be shaped as a right triangle with a hypotenuse extending out from a center of the insertion funnel entrance 254 upwards with respect to the insertion funnel entrance 254, such as towards sensing element 224. The insertion funnel 208 may include a right wall which may be positioned opposite a left wall of the insertion funnel 208. The right wall of the insertion funnel 208 may be composed of a rigid structure, such as plastic and/or other materials, without limitation. The right wall of the insertion funnel 208 may be shaped as a right triangle, square, rectangle, or other shape. In an instance the right wall of the insertion funnel 208 is positioned approximate the catch block 228. For instance, a right side of the right wall of the insertion funnel 208 may be contacting a side surface of the biasing mechanism 242, such as a left side surface of the biasing mechanism 242. In some embodiments, a right wall of the insertion funnel 208 may be positioned opposite a left wall of the insertion funnel 208. The left and right walls of the insertion funnel 208 may be spaced apart by, for instance and without limitation, 1 mm to 10 mm. In embodiments where the left and right walls of the insertion funnel 208 are right triangles, each hypotenuse of each wall may be extending towards a center of the first housing exit 257. The left and right walls of the first insertion funnel 208 may form the gap 244. The gap 244 may be slanted or turned at a radius of curvature, such that the sensing element 224 may be guided at an angle into the first housing exit 257. For instance, the sensing element 224 may be angled downwards at about 10 to about 90 degrees towards the first housing exit 257. The gap 244 may include a length of about, but not limited to, 2 mm. The gap 244 may include a width of about, but not limited to, 1.5 mm.

In a pre-deployment position shown in FIG. 2D, sliding member 216 and the sliding member 220 may be positioned proximate to one another. For instance, sliding member 216 and the sliding member 220 may be physically contacting one another, such as through one or more side surfaces of sliding member 216 and the sliding member 220. Sliding member 216 may have a distal end, such as the sensing element 224, positioned opposite a distal end of the sliding member 220, such as the cannula 232. In the pre-deployment position, the cannula 232 may extend at a downwards angle relative to the sliding member 220. For instance, the cannula 232 may be angled at about 15 degrees downwards from the sliding member 220 towards second insertion funnel entrance 255. The housing exit 255 may be the same as that of the first housing exit 257. The housing exit 255 may be positioned to a right side of the sliding member 220. In some embodiments, while in the pre-deployment position, a distal end of the cannula 232 may be positioned about 0.5 mm to about 5 mm, or about 3 mm up to 10 mm away from the housing exit 255. In some embodiments, a distal end of the cannula 232 may be positioned about 2 mm away from the housing exit 255.

Referring now to FIG. 2E, a side view of system 200 in an extended position is presented. In response to a triggering of biasing mechanism 242, the first arm 212A and/or the second arm 212B may push sliding member 216 and/or sliding member 220 in a direction L. The first arm 212A may be moved the direction L, by the biasing element 242 in a manner as described above with reference to FIG. 2C. The second arm 212B may apply force to sliding member 216. In some embodiments, sliding member 216 may apply the force received from the second arm 212B to a surface of the sliding member 220. During the movement in direction L, the cannula 232 of the second sliding element 220 may be pushed towards the housing exit 255. In the example, an angled position of the cannula 232 may allow for the cannula 232 to enter the housing exit 255 while the sliding member 220 is moved in direction L.

During the movement of sliding member 216 in the direction L, the wire 236 may stretch, fold, bend, and/or otherwise move with a surface of sliding member 216. For instance, a first or top end of the wire 236 may fold or bend horizontally in direction L towards a second or bottom end of the wire 236. In some embodiments, the wire 236 may fold or bend in half. In other embodiments, the wire 236 may fold or bend at various lengths relative to the wire 236, such as, for example, at about ¾ a total length of the wire 236. The wire 236 may be attached to sliding member 216 by an adhesive or another manner of attachment and be pulled in the direction L by sliding member 216.

Referring now to FIG. 2F, a retraction operation of system 200 is shown. The biasing element 242 as described above with reference to FIG. 2C may continue to rotate causing the first arm 212A and/or the second arm 212B to move in direction M. Direction M may include a linear direction, such as, away from and towards biasing mechanism 242. The second arm 212B may cause sliding member 216 to retract away from the sliding member 220. For instance, the second arm 212B may move in direction M causing sliding member 216 to also move in direction M. Sliding member 216 may continue in a path oriented to direction M until biasing mechanism 242 ceases to cause sliding member 216 to retract. The sensing element 224 of sliding member 216 may move through the insertion funnel 208 into the first housing exit 257. The insertion funnel 208 may guide the sensing element 224 into the first housing exit 257 as the second arm 212B moves in direction M. In some embodiments, the sensing element 224 may contact a top surface of a right wall of the insertion funnel 208. In some embodiments, the sensing element 224 may contact a bottom surface of a left wall of the insertion funnel 208. The sensing element 224 may be inserted up to, but not limited to, about 1 mm to about 30 mm, less than 1 mm, greater than 30 mm, and the like. For instance, the sensing element 224 may be inserted about 5.5 mm into the first housing exit 257. The biasing mechanism 242 and/or the second arm 212B may secure the sensing element 224 in the first housing exit 257 through the positioning arm first sliding member 116. The wire 236 may return from a folded and/or curved position to a relaxed position, such as that shown above in FIG. 2D. The wire 236 may maintain an electrical connection between sliding member 216 and the circuitry 253, which may allow the sensing element 224 now in a deployed position to communicate biological data with the circuitry 253.

In FIG. 2F, the sliding member 220 may be secured in a deployed position relative to the housing exit 255. The cannula 232 may be positioned in the housing exit 255. The sliding member 220 may secure the cannula 232 in housing exit 255, and the needle (indicated by the dashed line is shown retracted). For instance, the cannula 232 may be inserted and/or positioned approximately about 1 mm to about 30 mm, less than 1 mm, greater than 30 mm, and the like. For instance, the cannula 232 may be inserted at about mm or a depth into the housing exit 255 to deliver a liquid medication.

FIG. 2G, illustrates another embodiment of a dual insertion system. The dual insertion system 200G may include electric contact 248 in place of the wire 236 of the dual insertion system 200 shown in FIGS. 2A-2F. In the example, the electric contact 248 may be positioned on top of a surface of the circuitry 253a. The electric contact 248 may be positioned at a center of the circuitry 253a. The electric contact 248 may be in contact with the sensing element 224a and/or sliding member 216a in a pre-deployment position. The sensing element 224a may be similar to the sensing element 224 of FIGS. 2A-2F except for the form of the electrical connection between the sensing element 224a and the electrical contact 248. Similarly, sliding member 216a may be substantially similar to sliding member 216 except for the form of the electrical connection between the sensing element 224a and/or the electrical contact 216a. The electric contact 248 may establish an electrical contact with the sensing element 224a and/or sliding member 216a to enable the sensing element 224a to provide. The electric contact 248 may be elevated through bridge 262. The bridge 262 may be operable to extend the electric contact 248 and/or retract the electric contact 248 in a vertical position. For instance, the bridge 262 may extend the electric contact 248 by about 1 mm, which may allow the electric contact 248 to become in contact with the sensing element 224a and sliding member 216a. The bridge 262 may include, but is not limited to, an actuator, electromechanical motor, a biasing element, an elastic structure, or the like. The electric contact 248 may include a conductive material, such as, without limitation, copper, silver, and the like. The bridge 262 may be conductive, allowing an electrical connection between the electric contact 248 and the circuitry 253a.

Referring now to FIG. 2H, a deployment of system 200G is shown. The first arm 212A and/or the second arm 212B may push sliding member 216a and/or the sliding member 220 as described above with reference to FIG. 2E. In this example, the first and second arms 212A and 212B cause sliding member 216a to move in the direction Y which causes the cannula 232 to be inserted into the housing exit 255 and causes the sensing element 224a to align above the gap 244 of the insertion tunnel 208. During the deployment of the system 200G, the sliding member 220 may push or otherwise move cannula 232 into the housing exit 255. The cannula 232 may be angled, as described above with reference to FIG. 2E. The electric contact 248 may retract through the bridge 262 during a deployment of the system 200G. In an example, the electric contact 248 may retract at about 0.5 mm. towards the circuitry 253 away from the first arm 212A and the second arm 212B.

Referring now to FIG. 2I, a retraction of system 200G is presented. The first arm 212A and the second arm 212B may retract towards direction T. The second arm 212B may pull and/or otherwise move sliding member 216a towards direction T. Retraction of sliding member 216a towards direction T may be as described above with reference to FIG. 2H. Sliding member 116 may move in direction T towards which may align sliding member 216a with the electric contact 248. The electric contact 248 may contact or otherwise touch a surface of sliding member 216a, such as, without limitation, a bottom of sliding member 216a. During the deployment movement, the sensing element 224 may have entered the gap 244 and/or the housing exit 257, such as described above with reference to FIG. 2H. The sensing element 224a may collect and/or generate biological data and communicate the biological data to the circuit 253 through the electric contact 248. The sliding member 220 may stay in a deployed position with the cannula 232 inserted into the user's skin, as described above with reference to the embodiment of FIGS. 2A-2F.

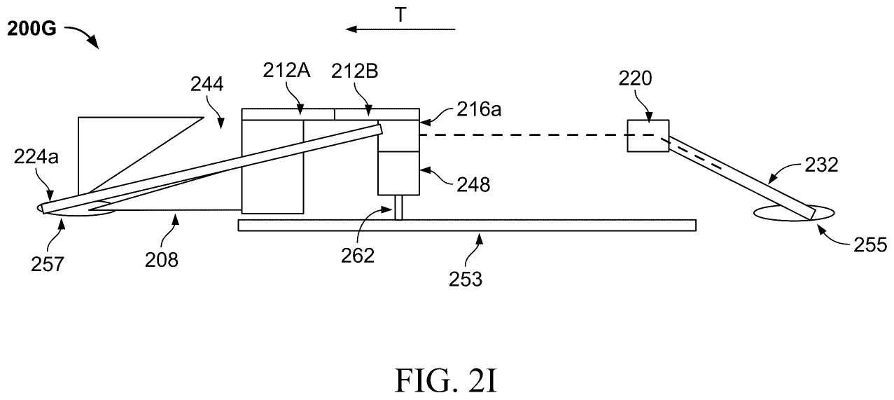

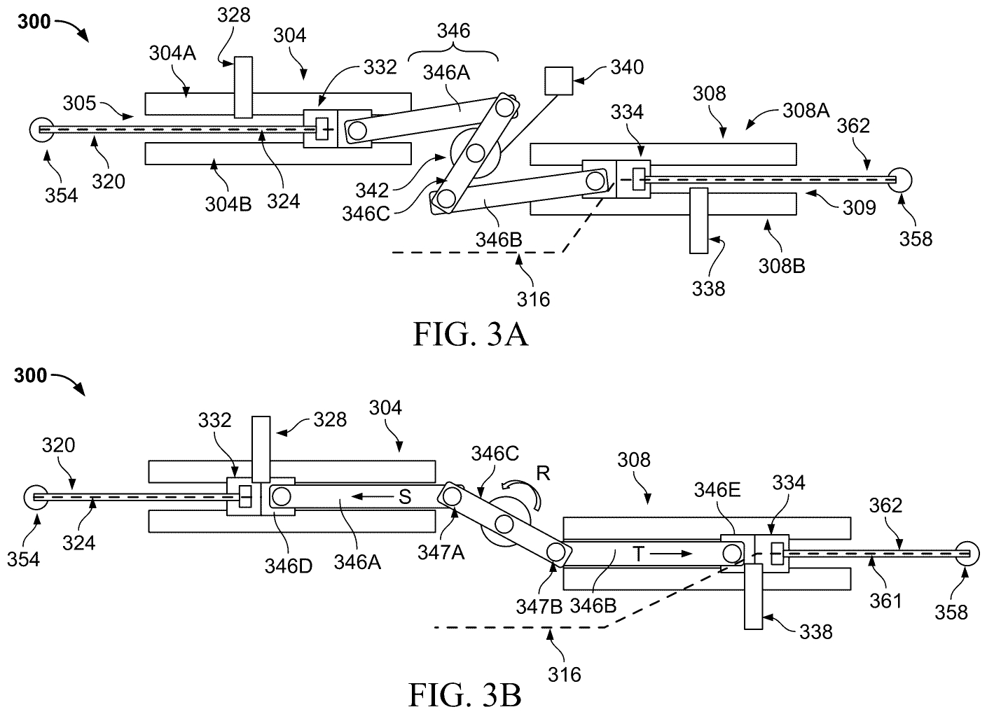

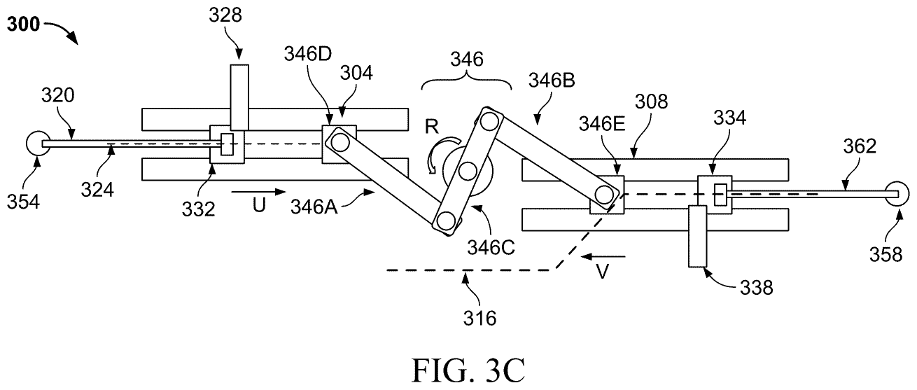

Referring now to FIG. 3A, another embodiment of an insertion system 300A is presented. System 300 may include positioning element 346. The positioning element 346 may include first arm 346A, second arm 346B, and/or third arm 346C. The first arm 346A may be positioned on a left side of the third arm 346C and the second arm 346B may be positioned on a right side of the third arm 346C, without limitation. A biasing mechanism 342 may be a spring or other form of force application element that is coupled to the positioning element 346 and is operable to apply a rotational force to, for example, the third arm 346C of the positioning element 346. The biasing mechanism 342 may be operable to store potential energy. A trigger 340 may be coupled to the biasing mechanism 342. The trigger 340 may be operable to release the potential energy stored by the biasing mechanism 342. The trigger 340 may be configured similar to and function in a similar manner as the trigger 240 as described above with reference to FIG. 2A. Each arm 346A and 346B of the positioning element 346 may be operable to move in a substantially linear direction, as discussed further below with reference to FIG. 3B, while arm 346C is operable to rotate. Additionally, the first arm 346A may be substantially aligned with a gap 305 of first rail 304.

The first rail 304 may include one or more linear structures 304A and 304B. In some embodiments, the first rail 304 may include a first set of two linear structures 304A and 304B that may be separated by a gap 305. The first sliding member 332 may be disposed on and/or housed within the gap 305 of the first set of rails o 304A and 304B. The sensing element 324 may extend distally from a left side of the first sliding member 332 towards housing exit 354. A first catching element 328 may be disposed on and/or otherwise positioned on a rail of the set of rails 304A and 304B. For instance, the first catching element 328 may be positioned on a top rail of one rails 304A or 304B, or both. The first catching element 328 may be made of rubber, plastic, and the like, without limitation. Catching element 328 may also serve to slow down insertion of cannula 362 during at least a portion of its insertion phase in a manner similar to that described above with reference to FIGS. 1B-1D.

The sensing element 320 may include first piercing element 320. The first piercing element 324 may include, without limitation, a steel trocar, needle, and/or other device. The first piercing element 324 may be positioned within an interior of the sensing element 320. For instance, the sensing element 320 may include a tube structure in which the first piercing element 324 may reside.

The second arm 346B may be positioned within a gap 309 of the second rail 308. The second rail 308 may be similar the first rail 304. The second rail 308 may include a first rail 308A and a second rail 308B.

The second rail 308 may guide the second sliding member 334 via the gap 309 between the rails 308A and 308B of the second rail 308. The second sliding member 334 may be connected to the cannula 362. The cannula 362 may be made of a flexible, stretchy material, such as a plastic tube. The cannula 362 may be positioned towards the second housing exit 358 and the sensing element 324 may be positioned towards the first housing exit 354, without limitation. The cannula 362 may be configured with a second piercing element 316 that also is fluidly coupled to a fluid pathway to a reservoir (shown in an earlier example) that holds a liquid medicament. The second piercing element 316 may be the same as that of the first piercing element 324. In some embodiments, the second piercing element 316 may include a needle, steel trocar, and the like, without limitation. The second rail 308 may include second catching element 338, which may be made of rubber, plastic, and the like, without limitation. Catching element 338 may also serve to slow down insertion of sensing element 324 during at least a portion of its insertion phase in a manner similar to that described above with reference to FIGS. 1B-1D.

Referring now to FIG. 3B, a deployment stage of system 300 is shown. Rotatable couplings 347A and 347B couple the arm 346C to respective arms 346A and 346B. The third arm 346C may rotate in direction R, which may cause the first arm 346A to move in direction S and the second arm 346B to move in direction T. The first arm 346A may move in direction S which may cause sliding member 346D to push the first sliding member 332 in the direction S. The first sliding member 332 when pushed by the first sliding member 346C causes the sensing element 320 move into the first housing exit 354. The second arm 346B may be moved in direction T through the rotation of third arm 346C in direction R. The second sliding member 334 may be pushed by sliding member 346E that is coupled to the second arm 346B the in direction T, which moves the cannula 362 towards the second housing exit 358. During a movement of the second arm 346B in direction T, the first piercing element 316 may be inserted into the second housing exit 358. Likewise, the first piercing element 324 may move in direction S into the first housing exit 354.

The first sliding member 332 may be held in position (and the sensing element 320 being held in its deployed position) by first catching element 328 and the second sliding member 334 may be held in position (and the cannula 362 being held in its deployed position). The “deployed position for the sensor,” for example, is when the sensor 320 is within the subcutaneous region of the patient's skin, and the “deployed position for the cannula 362,” for example, is when the cannula 362 is within the subcutaneous region of the patient's skin.

Referring now to FIG. 3C, a retraction stage of system 300 is shown. The third arm 346C may continue to dissipate energy and continue to move in direction R, which may move the first sliding member 346D in direction U and the second sliding member 346E in direction V. During the retraction stage, the first arm 346A may move the first piercing element 324 in direction U, out of the first housing exit 354. The first catching element 328 of the first rail 304 may provide resistance to movement of the first sliding member 332 in the direction U. The second arm 346B may move the second sliding member 346E in direction V which may move the second piercing element 316 in the direction V. The second catching element 338 may provide a resistance against a movement of the second sliding member 334 in the direction V. The second catching element 338 may secure the second sliding member 334 in a position, which may secure the cannula 362 in its deployed position.

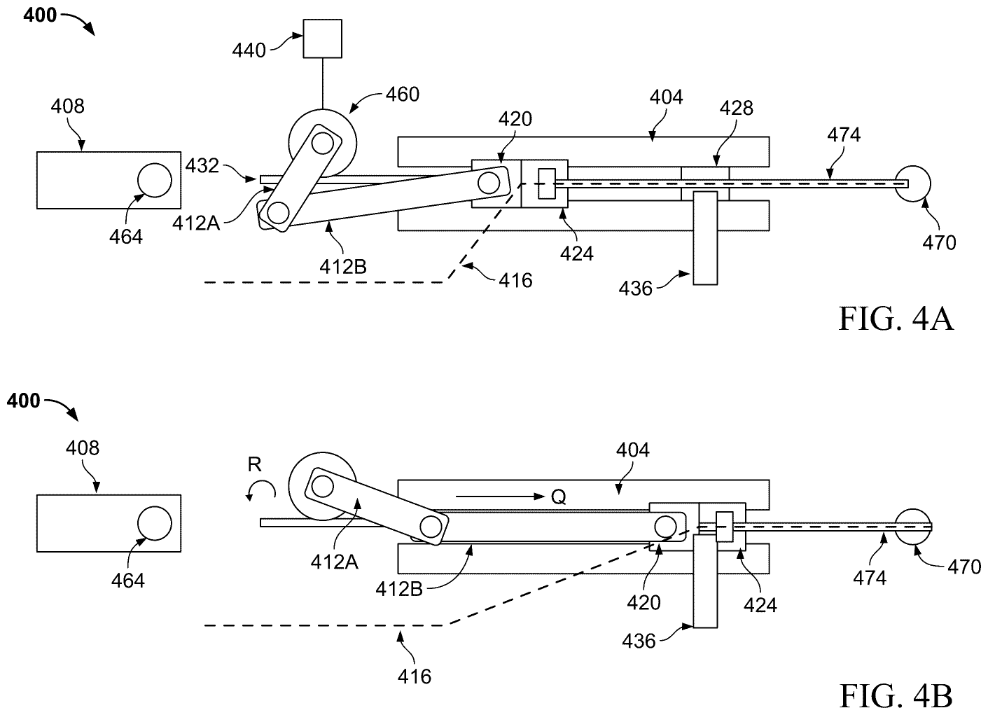

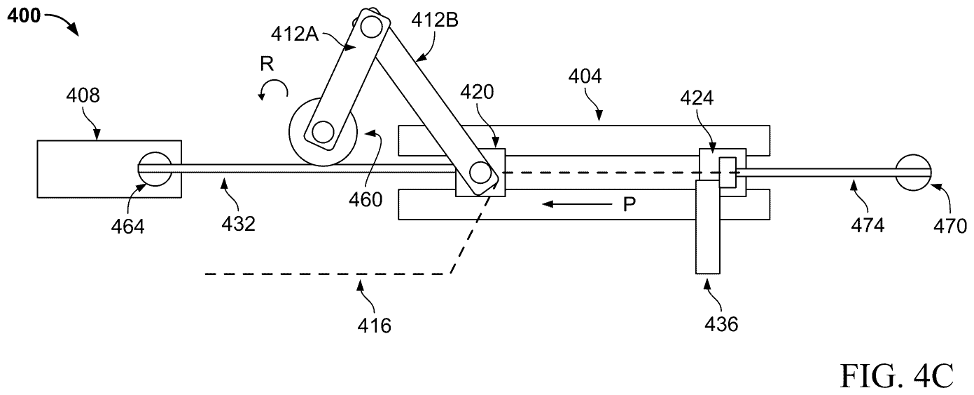

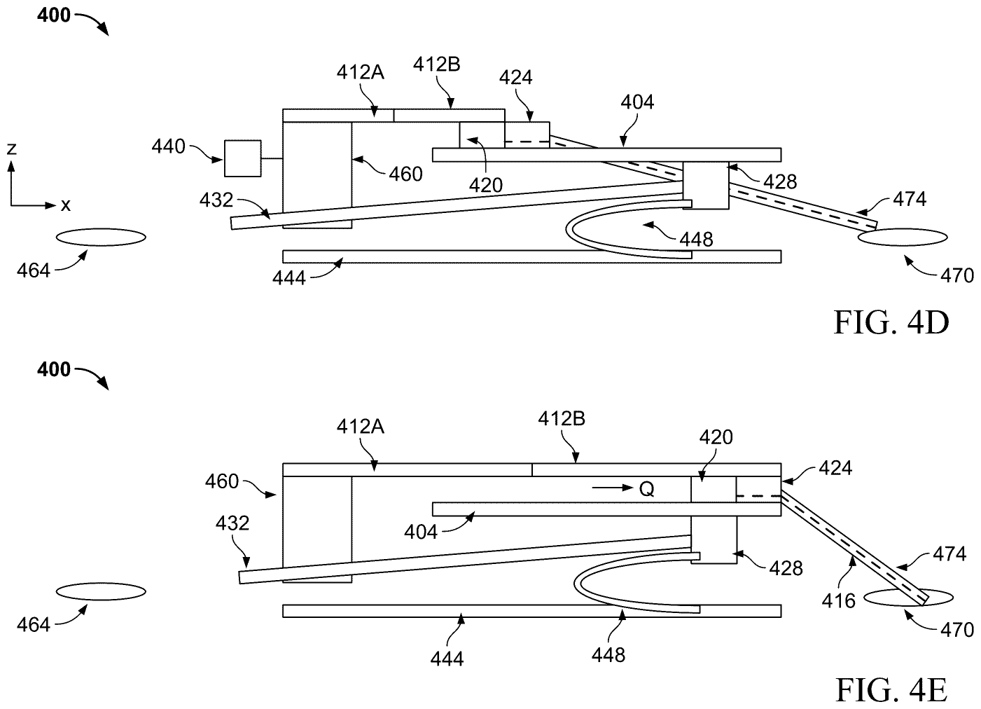

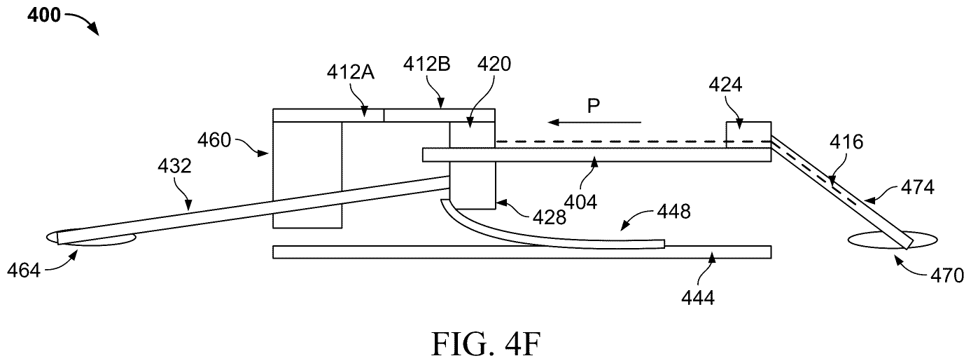

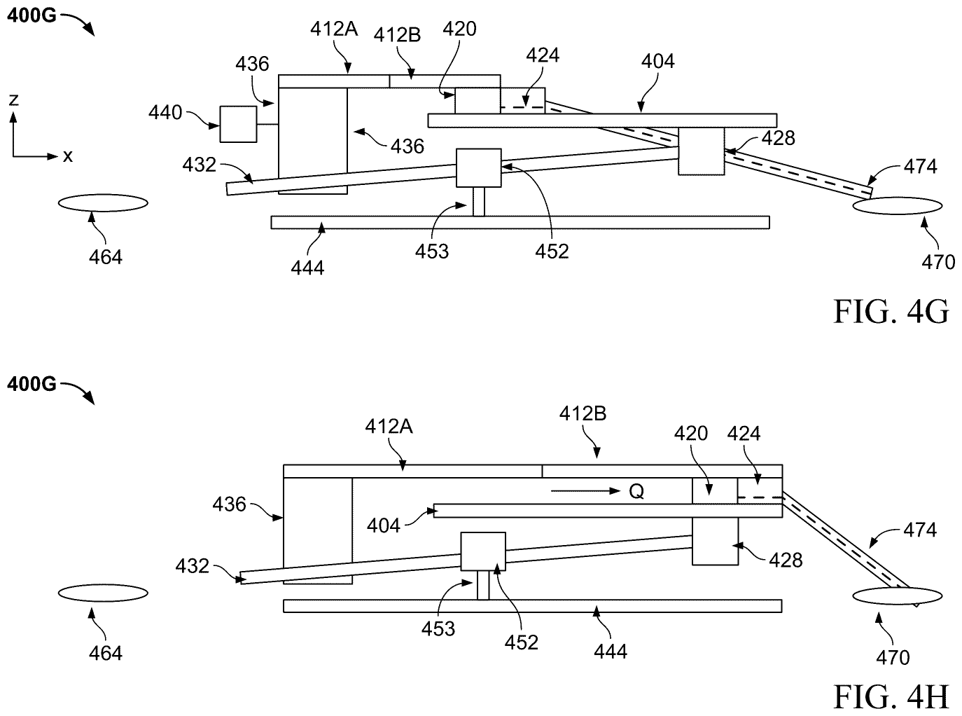

Referring now to FIG. 4A, a dual deployment system 400 having a single set of rails is presented. The system 400 illustrates a pre-deployment position. System 400 may include first arm 412A and/or second arm 412B. The first arm 412A may be connected to biasing element 460. The biasing element 460 may be the same as the biasing element 242 as described above with reference to FIG. 2A. The trigger 440 may active the biasing element 460. The trigger may include or be configured in the same manner as the trigger 240 as described above with reference to FIG. 2A. The system 400 may include the set of rails 404. The set of rails 404 may include one or more rigid linear structures. The set of rails 404 may include a set of two linear structures that have a gap between the two linear structures. The set of rails 404 may guide both the first sliding member 420 and the second sliding member 424. The first sliding member 420 may be oriented opposite or adjacent to the second sliding member 424. For instance, the first sliding member 420 may be coupled to sensing element 432 that may be extended outwards from the rail 404 towards insertion guide structure 408 and the second sliding member may include cannula 474 which may be extending outwards from the rail 404 towards the second housing exit 470. A first rail of the rails 404 may include catch block 436. The catch block 436 may be positioned at a center, end, and/or other length of the first rail. In some embodiments, the catch block 436 may be the same as the catch block 228 as described above with reference to FIG. 2A. The insertion guide structure 408 may include an external structure that supports an internal structure that may guide the sensing element 432 into the first housing exit 464. The sensor 428 may be configured to attach to the first sliding member 420, such as through one or more protrusions. The second sliding member 424 may be connected to and/or house the first piercing element 416. The first piercing element 416 may include or be similar to the first piercing element 214 as described above with reference to FIG. 2A.

Referring now to FIG. 4B a deployment stage of a dual deployment system 400 is presented. The biasing element 460 may be activated by the trigger 440. In some embodiments, the biasing element 460 may rotate in direction R. The biasing element 460 may cause the first arm 412A to rotate in direction R. The second arm 412B may push the first sliding member 420 towards the second sliding member 424, such as through direction Q. The sensor 432 may be attached to the first sliding member 420 during the movement. Of the second arm 412B in direction Q. The cannula 474 may be inserted into the second housing exit 470 through a movement of the second arm 412B in direction Q. In some embodiments, the first piercing element 416 may be inserted into the second housing exit 470.

Referring now to FIG. 4C, a retraction stage of a dual deployment system 400 is presented. The biasing element 460 may rotate in direction R which may cause the first arm 412A to rotate in direction R and the second arm 412B to move in direction P. The second arm 412B may move the first sliding member 420 in direction P. The sensing element 432 may be inserted into the insertion guide structure 408. The insertion guide structure 408 may include one or more guiding structures that may guide the sensing element 432 into the first housing exit 464. The first piercing element 416 may move in direction P through the second arm 412B, which may retract the first piercing element 416 from the second housing exit 470. The catching element 436 may oppose a movement in direction P, which may secure the second sliding member 424 in a deployed position.

Referring now to FIG. 4D, a side view of an embodiment of a dual deployment system 400 is illustrated. System 400 is shown in a pre-deployment position. The first arm 412A and the second arm 412B may be in a pre-deployment configuration. In some embodiments, the first sliding member 420 and the second sliding member 424 may be positioned adjacent to one another. The first sliding member 420 and the second sliding member 424 may be positioned on and/or between the set of rails 404. In some embodiments, the wire 448 may be connected to the interconnector 428. The interconnector 428 may include one or more protrusions, extending members, or the like, that may be placed on a side of one of the rails of the rails 404, for example, opposite the first sliding member 420 and the second sliding member 424. For instance, the first sliding member 420 and the second sliding member 424 may be positioned on a top surface of the rail 404 and the interconnector 428 may be positioned on a bottom surface of the rail 404.

The interconnector 428 may include one or more protrusions (not shown) that may extend from the bottom surface of a rail over/around a side of one rail of the rails 404 or into the gap between the set of rails of rails 404 to reach a top surface of the rail 404. For instance, and without limitation, the interconnector 428 may include an arm that may extend from the interconnector 428 to a top of the rail 404. In some embodiments, wire 448 may connect to interconnector 428 and/or circuitry 444. The wire 448 may include a conductive element such as, but not limited to, copper, aluminum, silver, and the like. The wire 448 may be similar to the wire 236 and function as described above with reference to FIG. 2D. In some embodiments, the wire 448 may have a first end and a second end. A first end of the wire 448 may be folded over horizontally towards a second end of the wire 448. The wire 448 may be folded in a positive direction across an x-axis. The wire 448 may be positioned on a right and/or center location of the circuitry 444. An electrical connection between the circuitry 444 and the interconnector 428 may be maintained through the wire 448. The circuitry 444 may be a processor or sensor circuitry that is operable to generate measurement data from physiological attributes detected by the sensor device 432.

Referring now to FIG. 4E, a side view of the deployment stage of the dual deployment system 400 is shown. In the example, the first arm 412A may rotate or otherwise move which may move the second arm 412B. The second arm 412B may move the first sliding member 420 and/or the second sliding member 424 in direction T. The set of rails 404 may provide structural support and/or guidance of a movement of the first sliding member 420 and the second sliding member 424 in direction T. The cannula 474 of the second sliding member 424 may be inserted into the second housing exit 470 during a movement of the second sliding member 424 in direction Q. The cannula 474 may be angled downward, such as at a degree of 15 relative to the rail 404, but is not limited to this angle. The interconnector 428 may physically attach to the first sliding member 420. For instance, an arm or other member of the interconnector 428 may clip, snap, hook, or otherwise connect the interconnector 428 to the first sliding member 420 over a side of the rail 404. The wire 448 may remain in a folded position as described above.

Referring now to FIG. 4F, a side view of a retraction of a dual deployment system is shown. The second arm 412B may retract or otherwise move in direction S through the first arm 412A moving in the direction S. The second arm 412B may move the first sliding member 420 in direction S which may move the sensing element 432 towards/into the first housing exit 464. The sensing element 432 may be angled downwards relative to the set of rails 404, such as by an angle of about 10-90 degrees, less than 10 degrees, greater than 90 degrees, or the like, without limitation. The second sliding member 424 may be secured in a deployment position, which may allow the cannula 474 to remain in the second housing exit 470. The piercing element 416 may move in direction P which may retract the piercing element 416 from the second housing exit 470. A fluid, such as a fluid medication, may be delivered to the second insertion funnel entrance 470 through the cannula 474 after the retraction of the piercing element 416, without limitation.

The wire 448 may extend in direction P through a pulling of the interconnector 428 in the direction P. The wire 448 may maintain an electrical connection between the circuitry 444 and the interconnector 428. The interconnector 428 may be connected to and/or in communication with the sensing element 432. The sensing element 432 may be positioned within the first housing exit 464 and collect and/or generate biological data which may be communicated through the wire 448 to the circuitry 444.

Referring now to FIG. 4G, a side view of a dual deployment system 400G in a pre-deployment stage is illustrated. The system 400G may be the same as that of the system 400D as described above with reference to FIG. 4D. The system 400G may include electric contact 452. The electric contact 452 may be the same as that of the electric contact 248 as described above with reference to FIG. 2G. The electric contact 452 may be made of, but not limited to, silver, copper, aluminum, and the like. In some embodiments, the electric contact 452 may be connected to and/or disposed on bridge 453. The bridge 453 may include an actuator, electromechanical motor, or other device that may extend and/or retract in a vertical direction. The bridge 453 may be conductive, which may provide an electric connection between the electric contact 453 and the circuitry 444. The electric contact 452 may be positioned on a center, left, or right location of the circuitry 444.

Referring now to FIG. 4H, a side view of a deployment stage of the dual deployment system 400G with the electrical contact 452 is shown. The biasing element 436 may activate, such as through the trigger 440 shown in FIG. 4G. The biasing element may rotate which may move the first sliding member 420 in direction S. The first sliding member 420 may push against or otherwise apply force to the second sliding member 424, which may cause the second sliding member 424 to move in direction S. The cannula 470 may move in direction S and may enter into the second housing exit 474. The interconnector 428 may connect with the first sliding member 420 as described above with reference to FIG. 4G.

Referring now to FIG. 4I, a side view of a retraction stage of the dual deployment system 400G is shown. The biasing element 436 may rotate which may cause the first arm 412A and/or the second arm 412B to move in direction P. The second arm 412B may move the sliding member 420 in direction P. The interconnector 428 may move with the first sliding member 420 through a physical attachment, such as a hook, loop, strap, and the like. The interconnector 428 may provide a connection between the electric contact 452 and the sensing element 432. The sensing element 432 may be pushed into or otherwise inserted into the first housing exit 464. The sensing element 464 may collect biological data that may be communicated to the circuitry 444 through the electric contact 452 that may be in contact with the interconnector 428. The second sliding member 424 may remain in a deployed position with the cannula 470 held in the second housing exit 474. The piercing element 416 may retract through a movement in direction P by the first sliding member 420 and/or the second arm 412B. The cannula 470 may be operable to deliver one or more fluids into the second housing exit 474 after retraction of the piercing element 416.

Referring now to FIG. 5, a block diagram of a medicament delivery system 500 is illustrated. In some examples, the medicament delivery system 500 is suitable for delivering a medicament such as insulin to a user in accordance with the disclosed embodiments. The medicament delivery system 500 may include a wearable medicament delivery device 502, a controller 504 and analyte sensor(s) 506. In addition, the medicament delivery system may interact with a computing device 532 via a network 508 as well as obtain or contribute to cloud-based services 510.

Still referring to FIG. 5, the wearable medicament delivery device 502 may be a wearable device that is worn on the body of the user. It may be similar a wearable medicament delivery and sensing system 100. The wearable medicament delivery device 502 may be directly coupled to a user (e.g., directly attached to the skin of the user via an adhesive, or the like at various locations on the user's body, such as thigh, abdomen, or upper arm). In an example, a surface of the wearable medicament delivery device 502 may include an adhesive to facilitate attachment to the skin of a user.

Still referring to FIG. 5, the wearable medicament delivery device 502 may include a processor 514. The processor 514 may be implemented in hardware, software, or any combination thereof. The processor 514 may, for example, be a microprocessor, a logic circuit, a field programmable gate array (FPGA), an application specific integrated circuit (ASIC) or a microprocessor coupled to a memory. The processor 514 may maintain a date and time as well as be operable to perform other functions (e.g., calculations or the like). The processor 514 may be operable to execute an AID application 526 stored in the memory 512 that enables the processor 514 to direct operation of the wearable medicament delivery device 502. The AID application 526 may control insulin delivery to the user per an AID algorithm. The memory 512 may store AID application settings for a user, such as specific factor settings, subjective insulin need parameter settings, and AID algorithm settings, such as maximum insulin delivery, insulin sensitivity settings, total daily insulin (TDI) settings and the like. The memory may also store data 529, such as medicament delivery dosages, blood glucose measurement values, ketone measurement levels, and the like.

Still referring to FIG. 5, the analyte sensor 506 may be operable to collect physiological condition data, such as the blood glucose measurement values and a timestamp, ketone levels, heart rate, blood oxygen levels and the like that may be shared with the wearable medicament delivery device 502, the controller 504 or both. For example, the communication circuitry 542 of the wearable medicament delivery device 502 may be operable to communicate with the analyte sensor 506 and the controller 504 as well as the devices 530, 533 and 534. The communication circuitry 542 may be operable to communicate via Bluetooth®, Wi-Fi, a near-field communication standard, a cellular standard, or any other wireless protocol. In an example, the wearable medicament delivery and sensing system 100 may be a combination of the wearable medicament delivery device 502 and the analyte sensor 506.

Still referring to FIG. 5, the input/output device(s) 545 may one or more of a microphone, a speaker, a vibration device, a display, a push button, a touchscreen display, a tactile input surface, or the like. The input/output device(s) 545 may be coupled to the processor 514 and may include circuitry operable to generate signals based on received inputs and provide the generated signals to the processor 514. In addition, the input/output device(s) 545 may be operable to receive signals from the processor 514 and, based on the received signals, generate outputs via a respective output device.

Still referring to FIG. 5, the wearable medicament delivery device 502 may include a reservoir 511. The reservoir 511 may be operable to store medicaments, medications, or therapeutic agents suitable for automated delivery, such as insulin, morphine, methadone, hormones, glucagon, glucagon-like peptide, GIP, blood pressure medicines, chemotherapy medicaments, combinations of medicaments, such as insulin and glucagon-like peptide, different peptides, or the like. A fluid path to the user may be provided via tubing and a needle/cannula (not shown). The fluid path may, for example, include tubing coupling the wearable medicament delivery device 502 to the user (e.g., via tubing coupling a needle or cannula to the reservoir 511). The wearable medicament delivery device 502 may be operable based on control signals from the processor 514 to expel the medicaments, medications, or therapeutic agents, such as insulin, from the reservoir 511 to deliver doses of the medicaments, medications, or therapeutic agents, such as the insulin, to the user via the fluid path. For example, the processor 514 by sending control signals to the pump 518 may be operable to cause insulin to be expelled from the reservoir 511.

Still referring to FIG. 5, there may be one or more communication links 598 with one or more devices physically separated from the wearable medicament delivery device 502 including, for example, a controller 504 of the user and/or a caregiver of the user and/or a sensor 506. The analyte sensor 506 may communicate with the wearable medicament delivery device 502 via a wireless communication link 531 and/or may communicate with the controller 504 via a wireless communication link 537. The communication links 531, 537, and 598 may include wired or wireless communication paths operating according to any known communications protocol or standard, such as Bluetooth, Wi-Fi, a near-field communication standard, a cellular standard, or any other wireless protocol.

Still referring to FIG. 5, the wearable medicament delivery device 502 may also include a user interface (UI) 516, such as an integrated display device for displaying information to the user, and in some embodiments, receiving information from the user. For example, the user interface 516 may include a touchscreen and/or one or more input devices, such as buttons, knob or a keyboard that enable a user to provide an input.

Still referring to FIG. 5, in addition, the processor 514 may be operable to receive data or information from the analyte sensor 506 as well as other devices, such as smart accessory device 530, fitness device 533 or another wearable device 534 (e.g., a blood oxygen sensor or the like), that may be operable to communicate with the wearable medicament delivery device 502. For example, fitness device 533 may include a heart rate sensor and be operable to provide heart rate information or the like.

Still referring to FIG. 5, the wearable medicament delivery device 502 may interface with a network 508. The network 508 may include a local area network (LAN), a wide area network (WAN) or a combination therein and operable to be coupled wirelessly to the wearable medicament delivery device 502, the controller, and devices 530, 533, and 534. A computing device 532 may be interfaced with the network 508, and the computing device may communicate with the insulin delivery device 502. The computing device 532 may be a healthcare provider device, a guardian's computing device, or the like through which a user's controller 504 may interact to obtain information, store settings, and the like. The AID application 520 may be operable to execute an AID algorithm and present a graphical user interface on the computing device 532 enabling the input and presentation of information related to the AID algorithm. The computing device 532 may be usable by a healthcare provider, a guardian of the user of the wearable medicament delivery device 502, or another user.

Still referring to FIG. 5, the medicament delivery system 500 may include an analyte sensor 506 for detecting the levels of one or more analytes of a user, such as blood glucose levels, ketone levels, other analytes relevant to a diabetic treatment program, or the like. The analyte level values detected may be used as physiological condition data and be sent to the controller 504 and/or the wearable medicament delivery device 502. The sensor 506 may be coupled to the user by, for example, adhesive or the like and may provide information or data on one or more medical conditions and/or physical attributes of the user. The sensor 506 may be a continuous glucose monitor (CGM), ketone sensor, or another type of device or sensor that provides blood glucose measurements that is operable to provide blood glucose concentration measurements. The sensor 506 may be physically separate from the wearable medicament delivery device 502 or may be an integrated component thereof. The analyte sensor 506 may provide the processor 514 and/or processor 519 with physiological condition data indicative of measured or detected blood glucose levels of the user. The information or data provided by the sensor 506 may be used to modify an insulin delivery schedule and thereby cause the adjustment of medicament delivery operations of the wearable medicament delivery device 502.

Still referring to FIG. 5, in the depicted example, the controller 504 may include a processor 519 and a memory 528. The controller 504 may be a special purpose device, such as a dedicated personal diabetes manager (PDM) device. The controller 504 may be a programmed general-purpose device that is a portable electronic device, such as any portable electronic device, smartphone, smartwatch, fitness device, tablet or the like including, for example, a dedicated processor, such as processor, a micro-processor or the like. The controller 504 may be used to program or adjust operation of the wearable medicament delivery device 502 and/or the sensor 506. The processor 519 may execute processes to manage a user's blood glucose levels and that control the delivery of the medicament or a therapeutic agent (e.g., a liquid medicament or the like as mentioned above) to the user. The processor 519 may also be operable to execute programming code stored in the memory 528. For example, the memory 528 may be operable to store an AID application 520 for execution by the processor 519. The AID application 520 may be responsible for controlling the wearable medicament delivery device 502, including the automatic delivery of insulin based on recommendations and instructions from the AID algorithm, such as those recommendations and instructions described herein.

Still referring to FIG. 5, the memory 528 may store one or more applications, such as an AID application 520, a voice control application 521, and data 539 which may be the same as, or substantially the same as those described above with reference to the insulin delivery device 502. In addition, the settings 521 may store information, such as medicament delivery history, blood glucose measurement values over a period of time, total daily insulin values, and the like. The memory 528 may be further operable to store data and/or computer programs 539 and the like. In addition, the memory may store AID settings and parameters, insulin treatment program history (such as insulin delivery history, blood glucose measurement value history and the like. Other parameters such as insulin-on-board (IOB) and insulin-to-carbohydrate ratio (ICR) may be retrieved from prior settings and insulin history stored in memory. For example, the AID application 520 may be operable to store the AID algorithm settings, such as blood glucose target set points, insulin delivery constraints, basal delivery rate, insulin delivery history, wearable medicament delivery device status, and the like. The memory 528 may also be operable to store data such as a food database for carbohydrate (or macronutrient) information of food components (e.g., grilled cheese sandwich, coffee, hamburger, brand name cereals, or the like). The memory 528 may be accessible to the AID application 520 and the voice control application 521.

Still referring to FIG. 5, the input/output device(s) 543 of the controller 504 may one or more of a microphone, a speaker, a vibration device, a display, a push button, a tactile input surface, touchscreen, or the like. The input/output device(s) 543 may be coupled to the processor 519 and may include circuitry operable to generate signals based on received inputs and provide the generated signals to the processor 519. In addition, the input/output device(s) 543 may be operable to receive signals from the processor 519 and, based on the received signals, generate outputs via one or more respective output devices, such as a speaker, a vibration device, or a display.

Still referring to FIG. 5, the controller 504 may include a user interface (UI) 523 for communicating visually with the user. The user interface 523 may include a display, such as a touchscreen, for displaying information provided by the AID application 520 or voice control application 521. The touchscreen may also be used to receive input when it is a touch screen. The user interface 523 may also include input elements, such as a keyboard, button, knob or the like. In an operational example, the user interface 523 may include a touchscreen display controllable by the processor 519 and be operable to present the graphical user interface, and in response to a received input (audio or tactile), the touchscreen display is operable present a graphical user interface related to the received input.

Still referring to FIG. 5, the controller 504 may interface via a wireless communication link of the wireless communication links 598 with a network, such as a LAN or WAN or combination of such networks that provides one or more servers or cloud-based services 510 via communication circuitry 522. The communication circuitry 522, which may include transceivers 527 and 525, may be coupled to the processor 519. The communication circuitry 522 may be operable to transmit communication signals (e.g., command and control signals) to and receive communication signals (e.g., via transceivers 527 or 525) from the wearable medicament delivery device 502 and the analyte sensor 506. In an example, the communication circuitry 522 may include a first transceiver, such as 525, that may be a Bluetooth transceiver, which is operable to communicate with the communication circuitry 522 of the wearable medicament delivery device 502, and a second transceiver, such as 527, that may be a cellular transceiver, a Bluetooth® transceiver, a near-field communication transceiver, or a Wi-Fi transceiver operable to communicate via the network 508 with computing device 532 or with cloud-based services 510. While two transceivers 525 and 527 are shown, it is envisioned that the controller 504 may be equipped more or less transceivers, such as cellular transceiver, a Bluetooth transceiver, a near-field communication transceiver, or a Wi-Fi transceiver.

Still referring to FIG. 5, the cloud-based services 510 may be operable to store user history information, such as blood glucose measurement values over a set period of time (e.g., days, months, years), a medicament delivery history that includes insulin delivery amounts (both basal and bolus dosages) and insulin delivery times, types of insulin delivered, indicated meal times, blood glucose measurement value trends or excursions or other user-related diabetes treatment information, specific factor settings including default settings, present settings and past settings, or the like.

Still referring to FIG. 5, other devices, like smart accessory device 530 (e.g., a smartwatch or the like), fitness device 533 and other wearable device 534 may be part of the medicament delivery system 500. These devices may communicate with the wearable medicament delivery device 502 to receive information and/or issue commands to the wearable medicament delivery device 502. These devices 530, 533 and 534 may execute computer programming instructions to perform some of the control functions otherwise performed by processor 514 or processor 519. These devices 530, 533 and 534 may include user interfaces, such as touchscreen displays for displaying information such as current blood glucose level, insulin on board, insulin deliver history, or other parameters or treatment-related information and/or receiving inputs. The display may, for example, be operable to present a graphical user interface for providing input, such as request a change in basal insulin dosage or delivery of a bolus of insulin. Devices 530, 533 and 534 may also have wireless communication connections with the sensor 506 to directly receive blood glucose level data as well as other data, such as user history data maintained by the controller 504 and/or the wearable medicament delivery device 502.

Still referring to FIG. 5, the user interface 523 may be a touchscreen display controlled by the processor 519, and the user interface 523 is operable to present a graphical user interface that offers an input of a subjective insulin need parameter usable by the AID application 520. The processor 519 may cause a graphical user interface to be presented on the user interface 523. Different examples of the graphical user interface may be shown with respect to other examples. The AID application 520 may generate instructions for the pump 518 to deliver basal insulin to the user or the like.

Still referring to FIG. 5, the processor 519 is also operable to collect physiological condition data related to the user from sensors, such as the analyte sensor 506 or heart rate data, for example, from the fitness device 533 or the smart accessory device 530. In an example, the processor 519 executing the AID algorithm may determine a dosage of insulin to be delivered based on the collected physiological condition of the user and a specific factor determined based on the subjective insulin need parameter. The processor 519 may output a control signal via one of the transceivers 525 or 527 to the wearable medicament delivery device 502. The outputted signal may cause the processor 514 to deliver command signals to the pump 518 to deliver an amount of related to the determined dosage of insulin in the reservoir 511 to the user based on an output of the AID algorithm. The processor 519 may also be operable to perform calculations regarding settings of the AID algorithm as discussed as herein. Modifications to the AID algorithm settings provided via the voice control application 521, such as by the examples described herein, may be stored in the memory 528.

Software related implementations of the techniques described herein may include, but are not limited to, firmware, application specific software, or any other type of computer readable instructions that may be executed by one or more processors. Hardware related implementations of the techniques described herein may include, but are not limited to, integrated circuits (ICs), application specific ICs (ASICs), field programmable arrays (FPGAs), and/or programmable logic devices (PLDs). In some examples, the techniques described herein, and/or any system or constituent component described herein may be implemented with a processor executing computer readable instructions stored on one or more memory components.

In addition, or alternatively, while the examples may have been described with reference to a closed loop algorithmic implementation, variations of the disclosed examples may be implemented to enable open loop use. The open loop implementations allow for use of different modalities of delivery of insulin such as smart pen, syringe or the like. For example, the disclosed AP application and algorithms may be operable to perform various functions related to open loop operations, such as the generation of prompts requesting the input of information such as weight or age. Similarly, a dosage amount of insulin may be received by the AP application or algorithm from a user via a user interface. Other open-loop actions may also be implemented by adjusting user settings or the like in an AP application or algorithm.

Some examples of the disclosed device may be implemented, for example, using a storage medium, a computer-readable medium, or an article of manufacture which may store an instruction or a set of instructions that, if executed by a machine (i.e., processor or microcontroller), may cause the machine to perform a method and/or operation in accordance with examples of the disclosure. Such a machine may include, for example, any suitable processing platform, computing platform, computing device, processing device, computing system, processing system, computer, processor, or the like, and may be implemented using any suitable combination of hardware and/or software. The computer-readable medium or article may include, for example, any suitable type of memory unit, memory, memory article, memory medium, storage device, storage article, storage medium and/or storage unit, for example, memory (including non-transitory memory), removable or non-removable media, erasable or non-erasable media, writeable or re-writeable media, digital or analog media, hard disk, floppy disk, Compact Disk Read Only Memory (CD-ROM), Compact Disk Recordable (CD-R), Compact Disk Rewriteable (CD-RW), optical disk, magnetic media, magneto-optical media, removable memory cards or disks, various types of Digital Versatile Disk (DVD), a tape, a cassette, or the like. The instructions may include any suitable type of code, such as source code, compiled code, interpreted code, executable code, static code, dynamic code, encrypted code, programming code, and the like, implemented using any suitable high-level, low-level, object-oriented, visual, compiled and/or interpreted programming language. The non-transitory computer readable medium embodied programming code may cause a processor when executing the programming code to perform functions, such as those described herein.

Certain examples of the present disclosure were described above. It is, however, expressly noted that the present disclosure is not limited to those examples, but rather the intention is that additions and modifications to what was expressly described herein are also included within the scope of the disclosed examples. Moreover, it is to be understood that the features of the various examples described herein were not mutually exclusive and may exist in various combinations and permutations, even if such combinations or permutations were not made express herein, without departing from the spirit and scope of the disclosed examples. In fact, variations, modifications, and other implementations of what was described herein will occur to those of ordinary skill in the art without departing from the spirit and the scope of the disclosed examples. As such, the disclosed examples are not to be defined only by the preceding illustrative description.

It is emphasized that the Abstract of the Disclosure is provided to allow a reader to quickly ascertain the nature of the technical disclosure. It is submitted with the understanding that it will not be used to interpret or limit the scope or meaning of the claims. In addition, in the foregoing Detailed Description, various features are grouped together in a single example for streamlining the disclosure. This method of disclosure is not to be interpreted as reflecting an intention that the claimed examples require more features than are expressly recited in each claim. Rather, as the following claims reflect, inventive subject matter lies in less than all features of a single disclosed example. Thus, the following claims are hereby incorporated into the Detailed Description, with each claim standing on its own as a separate example. In the appended claims, the terms “including” and “in which” are used as the plain-English equivalents of the respective terms “comprising” and “wherein,” respectively. Moreover, the terms “first,” “second,” “third,” and so forth, are used merely as labels and are not intended to impose numerical requirements on their objects.

The foregoing description of examples has been presented for the purposes of illustration and description. It is not intended to be exhaustive or to limit the present disclosure to the precise forms disclosed. Many modifications and variations are possible in light of this disclosure. It is intended that the scope of the present disclosure be limited not by this detailed description, but rather by the claims appended hereto. Future filed applications claiming priority to this application may claim the disclosed subject matter in a different manner and may generally include any set of one or more limitations as variously disclosed or otherwise demonstrated herein.

What is claimed is:

1. A medicament delivery system, comprising;
a housing comprising:
a medicament delivery device; and
an injection system, wherein the injection system includes:
a set of rails; and
a first sliding member operable to move on the set of rails;
a second sliding member operable to move on the set of rails; and
a positioning element, wherein the positioning element is configured to move the first sliding member in a first direction guided by the set of rails and move the second sliding member in a second direction guided by the set of rails.

2. The medicament delivery system of claim 1, wherein the positioning element comprises a biasing mechanism, a first arm and a second arm.

3. The medicament delivery system of claim 1, wherein the first sliding member is a sensor sliding member coupled to a sensing element.

4. The medicament delivery system of claim 3, wherein the sensing element includes a continuous glucose monitoring sensor.

5. The medicament delivery system of claim 1, wherein the second sliding member is a cannula sliding member fluidly coupled to a fluid reservoir.

6. The medicament delivery system of claim 5, wherein the fluid reservoir is located in the housing and contains a liquid medicament.

7. The medicament delivery system of claim 1, wherein the positioning element comprises a biasing mechanism, a first arm and a second arm, and the biasing mechanism when triggered is operable to cause the first arm and the second arm to deploy a needle/cannula coupled to the first sliding member and a sensing element coupled to the second sliding member.

8. The medicament delivery system of claim 7, wherein the biasing mechanism is further operable to retract a needle from the needle/cannula after the first sliding member is deployed, and retract a needle of the sensor device once the second sliding member is deployed.

9. A medicament delivery apparatus, comprising:
a medicament delivery device; and
a set of rails, wherein a gap is between individual rails of the set of rails;
a sensing element positioned adjacent to the gap of the set of rails;
an injection device positioned adjacent to the gap of the set of rails and positioned to mirror the sensing element;
a biasing mechanism coupled to a first sliding member, wherein the first sliding member is operable to move a cannula along the set of rails in a first direction and a sensing element along the set of rails in a second direction, opposite the first direction; and
an insertion funnel positioned opposite the set of rails, wherein the insertion funnel includes an insertion funnel entrance configured to allow a passage of the sensing element into the insertion funnel.

10. The medicament delivery apparatus of claim 9, wherein the biasing mechanism is configured to rotate in a clockwise or counterclockwise direction.

11. The medicament delivery apparatus of claim 9, wherein the sensing element is positioned in the insertion funnel entrance through the insertion funnel during a retraction of the first sliding member.

12. The medicament delivery apparatus of claim 9, wherein the injection device includes a fluid pathway fluidically connected to a liquid reservoir.

13. The medicament delivery apparatus of claim 12, wherein the liquid reservoir contains medicament.

14. The medicament delivery apparatus of claim 9, further comprising a catch block positioned on a rail of the set of rails, the catch block configured to secure the cannula sliding member in a position.

15. The medicament delivery apparatus of claim 9, wherein the sensing element includes a continuous glucose monitoring sensor.

16. The medicament delivery apparatus of claim 10, wherein a piercing element of the injection device is inserted into a housing exit upon an extension of the needle/cannula sliding member.

17. The medicament delivery apparatus of claim 9, wherein the insertion funnel comprises two angled surfaces opposite one another.

18. The medicament delivery apparatus of claim 9, further comprising a trigger in communication with the biasing mechanism, wherein the trigger is configured to activate the biasing mechanism.

19. The medicament delivery apparatus of claim 9, wherein the first sliding member includes a protrusion extending towards the gap of the set of rails and is configured to couple to a sensor sliding member.

20. A medicament delivery system, comprising;
a housing comprising:
a medicament delivery device, wherein the medicament delivery device includes a pump; and
an injection system connected to the pump, wherein the injection system includes:
a first set of rails; and
a second set of rails; and
a first sliding member operable to move on the first set of rails, the first sliding member including a sensor;
a second sliding member operable to move on the second set of rails, the second sliding member including a cannula connected to the pump; and
a positioning element, wherein the positioning element is configured to move the first sliding member in a first direction guided by the first set of rails and move the second sliding member in a second direction guided by the second set of rails.
